(19) United States
(12) Patent Application Publication
(10) Pub. No.: US 2025/0074916 A1
(43) Pub. Date: Mar. 6, 2025

(54) BRM TARGETING COMPOUNDS AND ASSOCIATED METHODS OF USE

(71) Applicant: Prelude Therapeutics Incorporated, Wilmington, DE (US)

(72) Inventors: Artem Shvartsbart, Kennett Square, PA (US); Andrew Combs, Kennett Square, PA (US); John A. Rose, Newark, DE (US)

(21) Appl. No.: 18/810,743

(22) Filed: Aug. 21, 2024

Related U.S. Application Data
(60) Provisional application No. 63/520,682, filed on Aug. 21, 2023.

(51) Int. Cl. C07D 487/14; A61K 31/55; C07D 471/14

(52) U.S. Cl. CPC C07D 487/14; A61K 31/55; C07D 471/14

(57) ABSTRACT

The disclosure is directed to compounds of Formula I

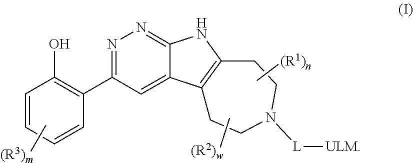

Pharmaceutical compositions comprising compounds of Formula I, as well as methods of their use and preparation, are also described.

### CROSS-REFERENCE TO RELATED APPLICATION

This application claims the benefit of U.S. Provisional Application No. 63/520,682, filed Aug. 21, 2023, the entirety of which is incorporated by reference herein.

### TECHNICAL FIELD

The description provides bifunctional compounds comprising a target protein binding moiety and a E3 ubiquitin ligase binding moiety, and associated methods of use. The bifunctional compounds are useful as modulators of targeted ubiquitination, especially with respect to Switch/Sucrose Non-Fermentable (SWI/SNF)-Related, Matrix-Associated, Actin-Dependent Regulator of Chromatin, Subfamily A, Member 2 (SMARCA2) (i.e., BRAHMA or BRM), which are degraded and/or otherwise inhibited by bifunctional compounds according to the present disclosure.

### BACKGROUND

The human SWItch/Sucrose Non-Fermentable (SWI/SNF) complexes are ATP-dependent chromatin remodelers. These large complexes play important roles in essential cellular processes, such as transcription, DNA repair and replication by regulating DNA accessibility.

Mutations in the genes encoding up to 20 canonical SWI/SNF subunits are observed in nearly 20% of all human cancers with the highest frequency of mutations observed in rhabdoid tumors, female cancers (including ovarian, uterine, cervical and endometrial), lung adenocarcinoma, gastric adenocarcinoma, melanoma, esophageal, and renal clear cell carcinoma.

SMARCA2 (BRM) and SMARCA4 (BRG1) are the subunits containing catalytic ATPase domains and they are essential for the function of SWI/SNF in perturbation of histone-DNA contacts, thereby providing access points to transcription factors and cognate DNA elements that facilitate gene activation and repression.

SMARCA2 and SMARCA4 shares a high degree of homology (up to 75%). SMARCA4 is frequently mutated in primary tumors (i.e., deleted or inactivated), particularly in lung cancer (12%), melanoma, liver cancer and pancreatic cancer. SMARCA2 is one of the top essential genes in SMARCA4-mutant (deleted) cancer cell line. This is because SMARCA4 deleted cancer cells exclusively rely on SMARCA2 ATPase activity for their chromatin remodeling activity for cellular functions such as cell proliferation, survival and growth. Thus, targeting SMARCA2 may be promising therapeutic approach in SMARCA4-related or deficient cancers (genetic synthetic lethality).

Previous studies have demonstrated the strong synthetic lethality using gene expression manipulation such as RNAi; downregulating SMARCA2 gene expression in SMARCA4 mutated cancer cells results in suppression of cancer cell proliferation. However, SMARCA2/4 bromodomain inhibitors (e.g., PFI-3) exhibit none to minor effects on cell proliferation inhibition [Vangamudi et al. Cancer Res 2015]. This phenotypic discrepancy between gene expression downregulation and small molecule-based approach lead us to investigating protein degradation bispecific molecules in SMARCA4 deficient cancers.

SMARCA2 is also reported to play roles in multiple myeloma expressing t(4; 14) chromosomal translocation [Chooi et al. Cancer Res abstract 2018]. SMARCA2 interacts with NSD2 and regulates gene expression such as PRL3 and CCND1. SMARCA2 gene expression downregulation with shRNA reduces cell cycle S phase and suppresses cell proliferation of t(4; 14) MM cells.

Therapeutic compounds that inhibit SMARCA2 and/or SMARCA4 are needed.

### SUMMARY

The present disclosure is directed to compounds of Formula (I):

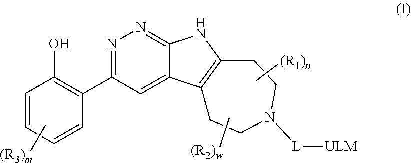

or a pharmaceutically acceptable salt thereof; wherein
each R¹ and R² is independently H, D, OR^{a}, C₁-C₈ alkoxy, C₁-C₈ alkyl, haloalkyl, —C₃-C₈ cycloalkyl, —C₃-C₁₀ cycloalkenyl, aryl, heteroaryl, heterocycloalkyl, heterocycloalkenyl, or (C₁-C₆-alkyl)-R^{e}; wherein said C₁-C₈ alkoxy, C₁-C₈ alkyl, haloalkyl, C₃-C₈ cycloalkyl, —C₃-C₁₀ cycloalkenyl, aryl, heteroaryl, heterocycloalkyl, heterocycloalkenyl, or (C₁-C₆-alkyl)-R^{e} are optionally substituted by 1-6 R^{f} groups; or
an R¹ and an R² may optionally be connected to form a 4-8 membered cycloalkyl or heterocycloalkyl ring;
R^{e} is C₃-C₈ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups;
each R^{f} is independently H, D, oxo, halogen, C₁-C₈ alkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)2NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —S(O)2R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocycloalkenyl group.
each R³ is independently H, D, halo, C₁₋₆ alkyl, haloalkyl, or C₃₋₆ cycloalkyl;
n is 1, 2, 3 or 4;
m is 1, 2, 3 or 4;
w is 1, 2, 3, or 4;
L is a linking group to ULM; and
ULM is a CRBN binding moiety or a VHL binding moiety.

Stereoisomers of the compounds of Formula I, and the pharmaceutical salts and stereoisomers thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. In some embodiments, alkyl groups of the disclosure are optionally substituted. Unless otherwise specified, in those embodiments wherein the alkyl group is substituted, the alkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the alkyl group is optionally substituted by 1-6 groups selected from D, halogen, —OH, —CN, —OR^{a}, —SR^{a}, —NR^{a}R^{d}, or NR^{c}R^{d}; or the alkyl group is optionally substituted by 1-6 R^{f} groups.

The term “halo” or halogen refers to chloro, fluoro, bromo, or iodo.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Cycloalkyl groups of the disclosure include monocyclic groups, as well as multicyclic groups such as bicyclic and tricyclic groups. In those embodiments having at least one multicyclic cycloalkyl group, the cyclic groups can share one common atom (i.e., spirocyclic). In other embodiments having at least one multicyclic cycloalkyl group, the cyclic groups share two common atoms (e.g., fused or bridged). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), spiro[3.3]heptanyl, bicyclo[3.3.0]octanyl, and the like. In some embodiments, cycloalkyl groups of the disclosure are optionally substituted. Unless otherwise specified, in those embodiments wherein the cycloalkyl group is substituted, the cycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkyl group is optionally substituted by 1-6 groups selected from D, halogen, —OH, —CN, —OR^{a}, —SR^{a}, —NR^{a}R^{d}, or NR^{c}R^{d}; or the cycloalkyl group is optionally substituted by 1-6 R^{f} groups.

The term “cycloalkenyl” when used alone or as part of a substituent group refers to monocyclic or multicyclic, partially saturated ring structure having from 3 to 10 carbon atoms (“C₃-C₁₀”), preferably from 3 to 6 carbon atoms (“C₃-C₆”). Cycloalkenyl groups of the disclosure include monocyclic groups, as well as multicyclic groups such as bicyclic and tricyclic groups. In those embodiments having at least one multicyclic cycloalkenyl group, the cyclic groups can share one common atom (i.e., spirocyclic). In other embodiments having at least one multicyclic cycloalkenyl group, the cyclic groups share two common atoms (e.g., fused or bridged). The term —C₃-C₆ cycloalkenyl refers to a cycloalkenyl group having between three and six carbon atoms. The cycloalkenyl group may be attached at any carbon atom of the partially saturated ring such that the result is a stable structure. Cycloalkenyl groups include groups in which the partially saturated ring is fused to an aryl group. Examples of cycloalkenyl groups include, for example, cyclopropenyl (C₃), cyclobutenyl (C₄), cyclopropenylmethyl (C₄), cyclopentenyl (C₅), cyclohexenyl (C₆), 1-methylcyclopropenyl (C₄), 2-methylcyclopentenyl (C₄), adamantenyl (C₁₀), spiro[3.3]heptenyl, bicyclo[3.3.0]octenyl, indanyl, and the like. In some embodiments, cycloalkenyl groups of the disclosure are optionally substituted. Unless otherwise specified, in those embodiments wherein the cycloalkenyl group is substituted, the cycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkenyl group is optionally substituted by 1-6 groups selected from D, halogen, —OH, —CN, —OR^{a}, —SR^{a}, —NR^{a}R^{d}, or NR^{c}R^{d}; or the cycloalkenyl group is optionally substituted by 1-6 R^{f} groups.

The term “heterocycloalkyl” when used alone or as part of a substituent group refers to any three to twelve membered monocyclic or multicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms (e.g., fused or bridged). The term —C₃-C₆ heterocycloalkyl refers to a heterocycloalkyl group having between three and six carbon ring atoms. The heterocycloalkyl group may be attached at any heteroatom or carbon atom of the group such that the result is a stable structure. Examples of heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl, diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, 3-azabicyclo[3.1.0]hexanyl,

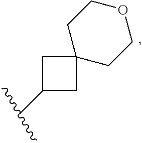 [SMILES: *C1CC2(CCOCC2)C1]

and the like. In some embodiments, heterocycloalkyl groups of the disclosure are optionally substituted. Unless otherwise specified, in those embodiments wherein the heterocycloalkyl group is substituted, the heterocycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkyl group is optionally substituted by 1-6 groups selected from D, halogen, —OH, —CN, —OR^{a}, —SR^{a}, —NR^{a}R^{d}, or NR^{c}R^{d}; or the heterocycloalkyl group is optionally substituted by 1-6 R^{f} groups.

The term “heterocycloalkenyl” when used alone or as part of a substituent group refers to any three to twelve membered monocyclic or multicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkenyl group, the cyclic groups can share one common atom (i.e., spirocyclic). In other embodiments having at least one multicyclic heterocycloalkenyl group, the cyclic groups share two common atoms (e.g., fused or bridged). The term —C₃-C₆ heterocycloalkenyl refers to a heterocycloalkenyl group having between three and six carbon atoms. The heterocycloalkenyl group may be attached at any heteroatom or carbon atom of the partially saturated ring such that the result is a stable structure. Heterocycloalkenyl groups include groups in which the partially saturated ring is fused to an aryl group, such as, for example isoindoline,

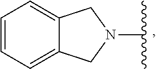 [SMILES: *N1Cc2ccccc2C1]

or in which the partially saturated ring is fused to a heteroaryl group, such as, for example, 6,7-dihydro-5H-pyrrolo[3,4-b]pyridine,

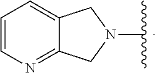 [SMILES: *N1Cc2cccnc2C1]

In some embodiments, heterocycloalkenyl groups of the disclosure are optionally substituted. Unless otherwise specified, in those embodiments wherein the heterocycloalkenyl group is substituted, the heterocycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkenyl group is optionally substituted by 1-6 groups selected from D, halogen, —OH, —CN, —OR^{a}, —SR^{a}, —NR^{a}R^{d}, or NR^{c}R^{d}; or the heterocycloalkenyl group is optionally substituted by 1-6 R^{f} groups.

The term “heterocyclic group,” or “heterocyclyl,” when used alone or as part of a substituent group, refers to a heterocycloalkyl group or a heterocycloalkenyl group.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. In some embodiments, heteroaryl groups of the disclosure are optionally substituted. Unless otherwise specified, in those embodiments wherein the heteroaryl group is substituted, the heteroaryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heteroaryl group is optionally substituted by 1-6 groups selected from D, halogen, —OH, —CN, —OR^{a}, —SR^{a}, —NR^{a}R^{d}, or NR^{c}R^{d}; or the heteroaryl group is optionally substituted by 1-6 R^{f} groups.

The term “aryl” when used alone or as part of a substituent group refers to a mono- or bicyclic-aromatic carbon ring structure. Aryl rings can include a total of 5, 6, 7, 8, 9, or 10 ring atoms. Examples of aryl groups include but are not limited to, phenyl, napthyl, and the like. In some embodiments, aryl groups of the disclosure are optionally substituted. Unless otherwise specified, in those embodiments wherein the aryl group is substituted, the aryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the aryl group is optionally substituted by 1-6 groups selected from D, halogen, —OH, —CN, —OR^{a}, —SR^{a}, —NR^{a}R^{d}, or NR^{c}R^{d}; or the aryl group is optionally substituted by 1-6 R^{f} groups.

When a range of carbon atoms is used herein, for example, C₁-C₆, all ranges, as well as individual numbers of carbon atoms are encompassed, for example, “C₁₋₃” includes C₁₋₃, C₁₋₂, C₂₋₃, C₁, C₂, and C₃. The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond.

The term “C₀-C₆alk” when used alone or as part of a substituent group refers to an aliphatic linker having 0, 1, 2, 3, 4, 5 or 6 carbon atoms. The term “—C₁alk-”, for example, refers to a —CH₂—. The term “—C₀alk-” refers to a bond.

Unless otherwise specified, in those embodiments wherein the —C₁-C₆alkyl, —C₁-C₁₀ alkyl, —C₁-C₈ alkoxy, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, heterocycloalkyl, (cycloalkyl)alkylcarbonyl, (cycloalkyl)carbonyl, (heterocyclyl) carbonyl, 3-5 membered cycloalkyl, 5-10 membered heteroaryl, alkoxy, alkyl, alkylcarbonyl, aralkyl, aralkylcarbonyl, aryl, arylcarbonyl, haloalkoxy, haloalkyl, napthyl, or phenyl groups are optionally substituted, they can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the —C₁-C₆alkyl, —C₁-C₁₀ alkyl, —C₁-C₈ alkoxy, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, heterocycloalkyl (cycloalkyl)alkylcarbonyl, (cycloalkyl)carbonyl, (heterocyclyl) carbonyl, 3-5 membered cycloalkyl, 5-10 membered heteroaryl, alkoxy, alkyl, alkylcarbonyl, aralkyl, aralkylcarbonyl, aryl, arylcarbonyl, haloalkoxy, haloalkyl, napthyl, or phenyl groups are optionally substituted by 1-6 groups selected from D, halogen, —OH, —CN, —OR^{a}, —SR^{a}, —NR^{a}R^{d}, or NR^{c}R^{d}; or the —C₁-C₆alkyl, —C₁-C₁₀ alkyl, —C₁-C₈ alkoxy, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, heterocycloalkyl (cycloalkyl)alkylcarbonyl, (cycloalkyl)carbonyl, (heterocyclyl) carbonyl, 3-5 membered cycloalkyl, 5-10 membered heteroaryl, alkoxy, alkyl, alkylcarbonyl, aralkyl, aralkylcarbonyl, aryl, arylcarbonyl, haloalkoxy, haloalkyl, napthyl, or phenyl groups are optionally substituted by 1-6 R^{f} groups.

In some embodiments, groups described herein as “optionally substituted” are unsubstituted.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Geometric isomers of the compounds of the present invention are described and may be isolated as a mixture of isomers or as separated isomeric forms.

Compounds of the invention may also include tautomeric forms. All tautomeric forms are encompassed.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 1 (1977) p. 1-19, each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., enantiomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

Where a range of values is provided, it is understood that each intervening value, to the tenth of the unit of the lower limit unless the context clearly dictates otherwise (such as in the case of a group containing a number of carbon atoms in which case each carbon atom number falling within the range is provided), between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure.

The following terms are used to describe the present disclosure. In instances where a term is not specifically defined herein, that term is given an art-recognized meaning by those of ordinary skill applying that term in context to its use in describing the present disclosure.

The articles “a” and “an” as used herein and in the appended claims are used herein to refer to one or to more than one (e.g., to at least one) of the grammatical object of the article unless the context clearly indicates otherwise. By way of example, “an element” means one element or more than one element.

The terms “co-administration” and “co-administering” or “combination therapy” refer to both concurrent administration (administration of two or more therapeutic agents at the same time) and time varied administration (administration of one or more therapeutic agents at a time different from that of the administration of an additional therapeutic agent or agents), as long as the therapeutic agents are present in the patient to some extent, preferably at effective amounts, at the same time. In certain preferred aspects, one or more of the present compounds described herein, are co-administered in combination with at least one additional bioactive agent, especially including an anticancer agent. In particularly preferred aspects, the co-administration of compounds results in synergistic activity and/or therapy, including anticancer activity.

The term “compound”, as used herein, unless otherwise indicated, refers to any specific chemical compound disclosed herein and includes tautomers, regioisomers, geometric isomers, and where applicable, stereoisomers, including optical isomers (enantiomers) and other stereoisomers (diastereomers) thereof, as well as pharmaceutically acceptable salts and derivatives, including prodrug and/or deuterated forms thereof where applicable, in context. Deuterated small molecules contemplated are those in which one or more of the hydrogen atoms contained in the drug molecule have been replaced by deuterium.

Within its use in context, the term compound generally refers to a single compound, but also may include other compounds such as stereoisomers, regioisomers and/or optical isomers (including racemic mixtures) as well as specific enantiomers or enantiomeric ally enriched mixtures of disclosed compounds. The term also refers, in context to prodrug forms of compounds which have been modified to facilitate the administration and delivery of compounds to a site of activity. It is noted that in describing the present compounds, numerous substituents and variables associated with same, among others, are described. It is understood by those of ordinary skill that molecules which are described herein are stable compounds as generally described hereunder.

The term “ubiquitin ligase” refers to a family of proteins that facilitate the transfer of ubiquitin to a specific substrate protein, targeting the substrate protein for degradation. For example, an E3 ubiquitin ligase protein that alone or in combination with an E2 ubiquitin-conjugating enzyme causes the attachment of ubiquitin to a lysine on a target protein, and subsequently targets the specific protein substrates for degradation by the proteasome. Thus, E3 ubiquitin ligase alone or in complex with an E2 ubiquitin conjugating enzyme is responsible for the transfer of ubiquitin to targeted proteins. In general, the ubiquitin ligase is involved in polyubiquitination such that a second ubiquitin is attached to the first; a third is attached to the second, and so forth. Polyubiquitination marks proteins for degradation by the proteasome. However, there are some ubiquitination events that are limited to mono-ubiquitination, in which only a single ubiquitin is added by the ubiquitin ligase to a substrate molecule. Mono-ubiquitinated proteins are not targeted to the proteasome for degradation, but may instead be altered in their cellular location or function, for example, via binding other proteins that have domains capable of binding ubiquitin. Further complicating matters, different lysines on ubiquitin can be targeted by an E3 to make chains. The most common lysine is Lys48 on the ubiquitin chain. This is the lysine used to make polyubiquitin, which is recognized by the proteasome.

As used herein, “Cereblon (CRBN) E3 Ubiquitin Ligase” refers to the substrate recognition subunit of the Cullin RING E13 ubiquitin ligase complexes. CRBN are one of the most popular E3 ligases recruited by bifunctional Proteolysis-targeting chimeras (PROTACs) to induce ubiquitination and subsequent proteasomal degradation of a target protein (Maniaci C. et al., Bioorg Med Chem. 2019, 27(12): 2466-2479).

As used herein, the term “heteroatom” is meant to include oxygen (O), nitrogen (N), sulfur (S) and silicon (Si). The nitrogen and sulfur can be in an oxidized form when feasible.

As used herein, the term “chiral” refers to molecules which have the property of non-superimposability of the mirror image partner, while the term “achiral” refers to molecules which are superimposable on their mirror image partner.

As used herein, the term “stereoisomers” refers to compounds which have identical chemical constitution but differ with regard to the arrangement of the atoms or groups in space, e.g., enantiomers, diastereomers, tautomers.

The term “patient” or “subject” is used throughout the specification to describe an animal, preferably a human or a domesticated animal, to whom treatment, including prophylactic treatment, with the compositions according to the present disclosure is provided. For treatment of those infections, conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

The term “effective” is used to describe an amount of a compound, composition or component which, when used within the context of its intended use, effects an intended result. The term effective subsumes all other effective amount or effective concentration terms, which are otherwise described or used in the present application.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

In one aspect, the disclosure is directed to a compound of Formula (I):

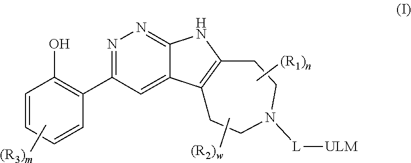

or a pharmaceutically acceptable salt thereof; wherein
each R¹ and R² is independently H, D, OR^{a}, C₁-C₈ alkoxy, C₁-C₈ alkyl, haloalkyl, —C₃-C₈ cycloalkyl, —C₃-C₁₀ cycloalkenyl, aryl, heteroaryl, heterocycloalkyl, heterocycloalkenyl, or (C₁-C₆-alkyl)-R^{e}; wherein said C₁-C₈ alkoxy, C₁-C₈ alkyl, haloalkyl, C₃-C₈ cycloalkyl, —C₃-C₁₀ cycloalkenyl, aryl, heteroaryl, heterocycloalkyl, heterocycloalkenyl, or (C₁-C₆-alkyl)-R^{e} are optionally substituted by 1-6 R^{f} groups; or
an R¹ and an R² may optionally be connected to form a 4-8 membered cycloalkyl or heterocycloalkyl ring;
R^{e} is C₃-C₈ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups;
each R^{f} is independently H, D, oxo, halogen, C₁-C₈ alkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)2NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR^{c})(OR^{d}), —S(O)2R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocycloalkenyl group.
each R³ is independently H, D, halo, C₁₋₆ alkyl, haloalkyl, or C₃₋₆ cycloalkyl;
n is 1, 2, 3 or 4;
m is 1, 2, 3 or 4;
w is 1, 2, 3, or 4;
L is a linking group to ULM; and
ULM is a CRBN binding moiety or a VHL binding moiety.

In some embodiments, each R¹ in Formula I is independently H, D, OR^{a}, C₁-C₈ alkoxy, C₁-C₈ alkyl, haloalkyl, —C₃-C₈ cycloalkyl, —C₃-C₈ cycloalkenyl, aryl, heteroaryl, heterocycloalkyl, heterocycloalkenyl, or (C₁-C₆-alkyl)-R^{e}; wherein said C₁-C₈ alkoxy, C₁-C₈ alkyl, haloalkyl, C₃-C₈ cycloalkyl, —C₃-C₁₀ cycloalkenyl, aryl, heteroaryl, heterocycloalkyl, heterocycloalkenyl, and (C₁-C₆-alkyl)-R^{e} are optionally substituted by 1-6 R^{f} groups.

In some embodiments, at least one R¹ in Formula I is H. In some embodiments, at least one R¹ in Formula I is D. In some embodiments, at least one R¹ in Formula I is OR^{a}. In some embodiments, at least one R¹ in Formula I is C₁-C₈ alkoxy. In other embodiments, at least one R¹ in Formula I is C₁-C₈ alkyl. In other embodiments, at least one R¹ in Formula I is haloalkyl. In other embodiments, at least one R¹ in Formula I is C₃-C₈ cycloalkyl. In other embodiments, at least one R¹ in Formula I is C₃-C₈ cycloalkenyl. In yet other embodiments, at least one R¹ in Formula I is aryl. In yet other embodiments, at least one R¹ in Formula I is heteroaryl. In yet other embodiments, the C₁-C₈ alkoxy, C₁-C₈ alkyl, haloalkyl, C₃-C₈ cycloalkyl, —C₃-C₁₀ cycloalkenyl, aryl, and heteroaryl are optionally substituted by 1-6 R^{f} groups.

In some embodiments, at least one R¹ in Formula I is H. In other embodiments, at least one R¹ in Formula I is C₁-C₈alkyl. In yet other embodiments, at least one R¹ in Formula I is methyl.

In some embodiments, at least one R¹ in Formula I is (C₁-C₆-alkyl)-R^{e} optionally substituted by 1-6 R^{f} groups. In some embodiments, R^{e} is azetidine optionally substituted by 1-6 R^{f} groups, pyrazole optionally substituted by 1-6 R^{f} groups, p-methoxybenzene or propyl. In some embodiments, R^{e} is pyrazole optionally substituted by 1-6 R^{f} groups. In other embodiments, R^{e} is p-methoxybenzene. In other embodiments, R^{e} is propyl.

In some embodiments, n in Formula I is 1, 2, 3 or 4. In some embodiments, n in Formula I is 1. In other embodiments, n in Formula I is 2. In yet other embodiments, n in Formula I is 3. In yet other embodiments, n in Formula I is 4.

In some embodiments, each R² in Formula I is independently H, D, OR^{a}, C₁-C₈ alkoxy, C₁-C₈ alkyl, haloalkyl, —C₃-C₈ cycloalkyl, —C₃-C₈ cycloalkenyl, aryl, heteroaryl, heterocycloalkyl, heterocycloalkenyl, or (C₁-C₆-alkyl)-R^{e}; wherein said C₁-C₈ alkoxy, C₁-C₈ alkyl, haloalkyl, C₃-C₈ cycloalkyl, —C₃-C₁₀ cycloalkenyl, aryl, heteroaryl, heterocycloalkyl, heterocycloalkenyl, and (C₁-C₆-alkyl)-R^{e} are optionally substituted by 1-6 R^{f} groups.

In some embodiments, at least one R² in Formula I is H. In some embodiments, at least one R² in Formula I is D. In some embodiments, at least one R² in Formula I is OR^{a}. In some embodiments, at least one R² in Formula I is C₁-C₈ alkoxy. In other embodiments, at least one R² in Formula I is C₁-C₈ alkyl. In other embodiments, at least one R² in Formula I is haloalkyl. In other embodiments, at least one R¹ in Formula I is C₃-C₈ cycloalkyl. In other embodiments, at least one R² in Formula I is C₃-C₈ cycloalkenyl. In yet other embodiments, at least one R² in Formula I is aryl. In yet other embodiments, at least one R² in Formula I is heteroaryl. In yet other embodiments, the C₁-C₈ alkoxy, C₁-C₈ alkyl, haloalkyl, C₃-C₈ cycloalkyl, —C₃-C₁₀ cycloalkenyl, aryl, and heteroaryl are optionally substituted by 1-6 R^{f} groups.

In some embodiments, at least one R² in Formula I is H. In other embodiments, at least one R² in Formula I is C₁-C₈alkyl. In yet other embodiments, at least one R² in Formula I is methyl.

In some embodiments, at least one R² in Formula I is (C₁-C₆-alkyl)-R^{e} optionally substituted by 1-6 R¹ groups. In some embodiments, R^{e} is azetidine optionally substituted by 1-6 R^{f} groups, pyrazole optionally substituted by 1-6 R^{f} groups, p-methoxybenzene or propyl. In other embodiments, R^{e} is pyrazole optionally substituted by 1-6 R^{f} groups. In other embodiments, R^{e} is p-methoxybenzene. In yet other embodiments, R^{e} is propyl.

In some embodiments, w in Formula I is 1, 2, 3 or 4. In some embodiments, w in Formula I is 1. In other embodiments, w in Formula I is 2. In yet other embodiments, w in Formula I is 3. In yet other embodiments, w in Formula I is 4.

In some embodiments, an R¹ and an R₂ in Formula I are connected to form a 4-8 membered cycloalkyl or heterocycloalkyl ring. In some embodiments, an R¹ and an R² in Formula I for a cyclopentyl ring. In other embodiments, an R¹ and an R² in Formula I for a cyclohexyl ring. In yet other embodiments, an R¹ and an R² in Formula I for a cycloheptyl ring. In yet other embodiments, an R¹ and an R² in Formula I for a cyclooctyl ring.

In some embodiments, each R¹ and R² is C₁₋₄ alkyl. In some embodiments, each R¹ and R² is methyl, ethyl or propyl.

In some embodiments, R^{e} in Formula I is C₃-C₈ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups.

In some embodiments, R^{e} in Formula I is C₃-C₈ cycloalkyl optionally substituted by 1-6 R^{f} groups. In some embodiments, R^{e} in Formula I is heterocycloalkyl optionally substituted by 1-6 R^{f} groups. In some embodiments, the heterocycloalkyl is attached to (C₁-C₆-alkyl) through a carbon atom of the heterocycloalkyl group. In some embodiments, the heterocycloalkyl is attached to (C₁-C₆-alkyl) through a sulfur atom of the heterocycloalkyl group. In other embodiments, R^{e} in Formula I is cycloalkenyl optionally substituted by 1-6 R^{f} groups. In other embodiments, R^{e} in Formula I is heterocycloalkenyl optionally substituted by 1-6 R^{f} groups. In other embodiments, the heterocycloalkenyl is attached to (C₁-C₆-alkyl) through a carbon atom of the heterocycloalkenyl group. In other embodiments, the heterocycloalkenyl is attached to (C₁-C₆-alkyl) through a sulfur atom of the heterocycloalkenyl group. In yet other embodiments, R^{e} in Formula I is aryl optionally substituted by 1-6 R^{f} groups. In yet other embodiments, R^{e} in Formula I is heteroaryl optionally substituted by 1-6 R^{f} groups.

In some embodiments, R^{e} in Formula I is azetidine or piperidine optionally substituted by 1-6 R^{f} groups, pyrazole optionally substituted by 1-6 R^{f} groups, phenyl optionally substituted by 1-6 R^{f} groups or cycloalkyl optionally substituted by 1-6 R^{f} groups. In some embodiments, R^{e} in Formula I is azetidine optionally substituted by 1-6 R^{f} groups. In some embodiments, R^{e} in Formula I is piperidine optionally substituted by 1-6 R^{f} groups. In some embodiments, R^{e} in Formula I is phenyl optionally substituted by 1-6 R^{f} groups. In some embodiments, R^{e} in Formula I is cycloalkyl optionally substituted by 1-6 R^{f} groups.

In some embodiments, each R^{f} in Formula I is independently H, D, oxo, halogen, C₁-C₈ alkoxy, C₁-C₈ alkyl, haloalkyl, —OH, —CN, —NO2, —C2-C6 alkenyl, —C2-C6 alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)2NR^{c}R^{d}, —S(O)(═NR^{b})R^{b}, —SF5, —P(O)R^{b}R^{b}, —P(O)(OR^{b})(OR^{b}), —B(OR^{c})(OR^{d}), —S(O)2R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In some embodiments, R^{f} in Formula I is H. In some embodiments, R^{f} in Formula I is D. In some embodiments, R^{f} in Formula I is oxo. In some embodiments, R^{f} in Formula I is halogen. In some embodiments, R^{f} in Formula I is C₁-C₈ alkoxy. In some embodiments, R^{f} in Formula I is C₁-C₈ alkyl. In some embodiments, the C₁-C₈ alkyl is optionally substituted by 1-6 groups selected from D, halogen, —OH, —CN, —OR^{a}, —SR^{a}, —NR^{a}R^{d}, or NR^{c}R^{d}. In some embodiments, R^{f} in Formula I is haloalkyl. In some embodiments, R^{f} in Formula I is —OH. In some embodiments, R^{f} in Formula I is —CN. In some embodiments, R^{f} in Formula I is —NO2. In some embodiments, R^{f} in Formula I is —C₂-C₆ alkenyl. In some embodiments, R^{f} in Formula I is —C2-C6 alkynyl. In some embodiments, R^{f} in Formula I is aryl. In some embodiments, R^{f} in Formula I is heteroaryl. In some embodiments, R^{f} in Formula I is cycloalkyl. In other embodiments, R^{f} in Formula I is cycloalkenyl. In other embodiments, R^{f} in Formula I is heterocycloalkyl. In other embodiments, R^{f} in Formula I is heterocycloalkenyl. In other embodiments, R^{f} in Formula I is —OR^{a}. In other embodiments, R^{f} in Formula I is —SR^{a}. In other embodiments, R^{f} in Formula I is —NR^{c}R^{d}. In other embodiments, R^{f} in Formula I is —NR^{a}R^{c}. In other embodiments, R^{f} in Formula I is —C(O)R^{b}. In other embodiments, R^{f} in Formula I is —OC(O)R^{b}. In other embodiments, R^{f} in Formula I is —C(O)OR^{b}. In other embodiments, R^{f} in Formula I is —C(O)NR^{c}R^{d}. In yet other embodiments, R^{f} in Formula I is —S(O)R^{b}. In yet other embodiments, R^{f} in Formula I is —S(O)₂NR^{c}R^{d}. In yet other embodiments, R^{f} in Formula I is —S(O)(═NR^{b})R^{b}. In yet other embodiments, R^{f} in Formula I is —SF5. In yet other embodiments, R^{f} in Formula I is —P(O)R^{b}R^{b}. In yet other embodiments, R in Formula I is —P(O)(OR^{b})(OR^{b}). In yet other embodiments, R¹ in Formula I is —B(OR^{c})(OR^{d}). In yet other embodiments, R^{f} in Formula I is —S(O)2R^{b}. In yet other embodiments, R^{f} in Formula I is —C(O)NR^{b}OR^{b}. In yet other embodiments, R^{f} in Formula I is —S(O)₂OR^{b}. In yet other embodiments, R in Formula I is —OS(O)₂OR^{b}. In yet other embodiments, R^{f} in Formula I is —OPO(OR^{b})(OR^{b}).

In some embodiments, m in Formula I is 1, 2, 3 or 4. In some embodiments, m in Formula I is 1. In other embodiments, m in Formula I is 2. In yet other embodiments, m in Formula I is 3. In yet other embodiments, m in Formula I is 4.

In some embodiments, each R³ in Formula I is independently H, D, halo, C₁₋₆ alkyl, haloalkyl, or C₃₋₆ cycloalkyl. In some embodiments, at least one R³ in Formula I is H. In some embodiments, at least one R³ in Formula I is D. In other embodiments, at least one R³ in Formula I is C₁₋₆ halo. In other embodiments, at least one R³ in Formula I is C₁₋₆ alkyl. In other embodiments, at least one R³ in Formula I is haloalkyl. In yet other embodiments, at least one R³ in Formula I is C₃₋₆ cycloalkyl.

In some embodiments, at least one R³ in Formula I is F.

In some embodiments, each R^{a} in Formula I is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl.

In some embodiments, R^{a} in Formula I is H. In some embodiments, R^{a} in Formula I is D. In some embodiments, R^{a} in Formula I is —C(O)R^{b}. In some embodiments, R^{a} in Formula I is —C(O)OR^{c}. In some embodiments, R^{a} in Formula I is —C(O)NR^{c}R^{d}. In some embodiments, R^{a} in Formula I is —C(═NR^{b})NR^{b}R^{c}. In some embodiments, R^{a} in Formula I is C(═NOR^{b})NR^{b}R^{c}. In some embodiments, R^{a} in Formula I is —C(═NCN)NR^{b}R^{c}.

In other embodiments, R^{a} in Formula I is —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, and the like. In yet other embodiments, R^{a} in Formula I is —C₁-C₁₀alkyl, —C₂-C₁₀ alkenyl, —C₂-C₁₀ alkynyl, aryl, cycloalkyl, cycloalkenyl, heteroaryl, heterocycloalkyl, heterocycloalkenyl, and the like.

In some embodiments, each R^{b} in Formula I is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl.

In some embodiments, R^{b} in Formula I is H. In some embodiments, R^{b} in Formula I is D. In some embodiments, R^{b} in Formula I is —C₁-C₆ alkyl. In some embodiments, R^{b} in Formula I is —C₂-C₆ alkenyl. In some embodiments, R^{b} in Formula I is —C₂-C₆ alkynyl. In other embodiments, R^{b} in Formula I is aryl. In other embodiments, R^{b} in Formula I is cycloalkyl. In other embodiments, R^{b} in Formula I is cycloalkenyl. In other embodiments, R^{b} in Formula I is heteroaryl. In other embodiments, R^{b} in Formula I is heterocycloalkyl. In other embodiments, R^{b} in Formula I is heterocycloalkenyl.

In some embodiments, each R^{c} or R^{d} in Formula I is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl.

In some embodiments, R^{c} or R^{d} in Formula I is H. In some embodiments, R^{c} or R^{d} in Formula I is D. In some embodiments, R^{c} or R^{d} in Formula I is —C₁-C₁₀ alkyl. In some embodiments, R^{c} or R^{d} in Formula I is —C₂-C₆ alkenyl. In some embodiments, R^{c} or R^{d} in Formula I is —C₂-C₆ alkynyl. In other embodiments, R^{c} or R^{d} in Formula I is —OC₁-C₆alkyl. In other embodiments, R^{c} or R^{d} in Formula I is —O-cycloalkyl. In other embodiments, R^{c} or R^{d} in Formula I is aryl. In other embodiments, R^{c} or R^{d} in Formula I is cycloalkyl. In other embodiments, R^{c} or R^{d} in Formula I is cycloalkenyl. In other embodiments, R^{c} or R^{d} in Formula I is heteroaryl. In other embodiments, R^{c} or R^{d} in Formula I is heterocycloalkyl. In other embodiments, R^{c} or R^{d} in Formula I is heterocycloalkenyl.

In yet other embodiments, R^{c} and R^{d} in Formula I, together with the atom to which they are both attached, form a monocyclic or multicyclic heterocycloalkyl, or a monocyclic or multicyclic heterocycloalkenyl group. In yet other embodiments, R^{c} and R^{d} in Formula I form a monocyclic heterocycloalkyl. In yet other embodiments, R^{c} and R^{d} in Formula I form a multicyclic heterocycloalkyl. In yet other embodiments, R^{c} and R^{d} in Formula I form a monocyclic heterocycloalkenyl group. In yet other embodiments, R^{c} and R^{d} in Formula I form a multicyclic heterocycloalkenyl group.

According to the disclosure, and in some embodiments, ULM is a small molecule E3 Ubiquitin Ligase binding moiety that binds a Cereblon E3 Ubiquitin Ligase. In some embodiments, ULM is a moiety as described herein.

Chemical moieties that are used to link to ULM moieties are known in the art. These moieties are sometimes referred to as “linkers” in the art. In some embodiments, L in Formula I is a chemical moiety that is used to link to ULM that is known in the art.

In some embodiments, L in Formula I is a chemical moiety that is used to link to ULM as described in U.S. Patent Application Publication No. 2019/0300521, the entirety of which is incorporated by reference herein.

In other embodiments, L in Formula I is a chemical moiety that is used to link to ULM as described in U.S. Patent Application Publication No. 2019/0255066, the entirety of which is incorporated by reference herein.

In other embodiments, L in Formula I is a chemical moiety that is used to link to ULM as described in WO 2019/084030, the entirety of which is incorporated by reference herein.

In other embodiments, L in Formula I is a chemical moiety that is used to link to ULM as described in WO 2019/084026, the entirety of which is incorporated by reference herein.

In some embodiments, L in Formula I is a chemical structural unit represented by the formula:

-(A)_{q}-,

wherein:
q is an integer from 1 to 14;
each A is independently selected from the group consisting of CR^{1a}R^{1b}, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄ O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}NR^{1c}C(═NCN), NR^{1c}C(═CNO₂)NR^{1d}, 3-11 membered cycloalkyl, optionally substituted with 1-6 R^{1a} or R^{1b} groups, 3-11 membered heterocyclyl optionally substituted with 1-6 R^{1a} or R^{1b} groups, aryl optionally substituted with 1-6 R^{1a} or R^{1b} groups, heteroaryl optionally substituted with 1-6 R^{1a} or R^{1b} groups,
and R^{1a}, R^{1b}, R^{1c}, R^{1d} and R^{1e} are each independently, —H, D, -halo, —C₁-C₈alkyl, —C₁-C₆haloalkyl, —O—C₁-C₈alkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂— heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)-CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; or where the context permits, R^{1a} or R^{1b}, are linked to other groups, or to each other, to form a cycloalkyl and/or a heterocyclyl moiety, optionally substituted with 1-4 R^{1e} groups.

In these embodiments, q represents the number of connected A groups. For example, when q=1, -(A)_{q}- is -A₁-; when q=2, -(A)_{q}- is -A₁-A₂-; when q=3, -(A)_{q}- is -A₁-A₂-A₃-; when q=4, -(A)_{q}- is -A₁-A₂-A₃-A₄-; when q=5, -(A)_{q}- is -A₁-A₂-A₃-A₄-A₅-; when q=6, -(A)_{q}- is -A₁-A₂-A₃-A₄-A₅-A₆-; when q=7, -(A)_{q}- is -A₁-A₂-A₃-A₄-A₅-A₆-A₇-; when q=8, - (A)_{q}- is -A₁-A₂-A₃-A₄-A₅-A₆-A₇-A₈-; when q=9, -(A)_{q}- is -A₁-A₂-A₃-A₄-A₅-A₆-A₇-A₈-A₉-; when q=10, -(A)_{q}- is -A₁-A₂-A₃-A₄-A₅-A₆-A₇-A₈-A₉-A₁₀-; when q=11, -(A)_{q}- is -A₁-A₂-A₃-A₄-A₅-A₆-A₇-A₈-A₉-A₁₀-A₁₁-; when q=12, -(A)_{q}- is -A₁-A₂-A₃-A₄-A₅-A₆-A₇-A₈-A₉-A₁₀-A₁₁-A₁₂- ; when q=13, -(A)_{q}- is -A₁-A₂-A₃-A₄-A₅-A₆-A₇-A₈-A₉-A₁₀-A₁₁-A₁₂-A₁₃-; and when q=14, -(A)_{q}- is - A₁-A₂-A₃-A₄-A₅-A₆-A₇-A₈-A₉-A₁₀-A₁₁-A₁₂-A₁₃-A₁₄-.

In some embodiments, q=4 and Y in Formula IA is a chemical moiety represented by the formula: -A₁-A₂-A₃-A₄-, wherein each of A₁₋₄ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄ NR^{1c}(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cycloalkyl, C₃₋₈ heterocycloalkyl, aryl, or heteroaryl.

In other embodiments, q=3 and Y in Formula IA is a chemical moiety represented by the formula: -A₁-A₂-A₃-, wherein each of A₁₋₃ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C≡C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cycloalkyl, C₃₋₈ heterocycloalkyl, aryl, or heteroaryl.

In other embodiments, q=2 and L in Formula I is a chemical moiety represented by the formula: -A₁-A₂-, wherein each of A₁₋₂ is independently selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C—C≡C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cycloalkyl, C₃₋₈ heterocycloalkyl, aryl, or heteroaryl.

In other embodiments, q=1 and L in Formula I is a chemical moiety represented by the formula: -A₁-, wherein A₁ is selected from the group consisting of O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})_{1- 4}, —(CR^{1a}R^{1b})₁₋₄S(CR^{1a}R^{1b})₁₋₄, (CR^{1a}R^{1b})₁₋₄NR^{1c}(CR^{1a}R^{1b})₁₋₄, optionally substituted 3-11 membered cycloalkyl, 3-11 membered heterocyclyl, aryl, and heteroaryl;
wherein R^{1a} and R^{1b} are each independently selected from the group consisting of —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and
R^{1c} and R^{1d} are each independently selected from the group consisting of H, D, optionally substituted C₁₋₄ alkyl, C₃₋₈ cycloalkyl, C₃₋₈ heterocycloalkyl, aryl, or heteroaryl.

In some embodiments, L in Formula I is a covalent bond, 3-11 membered cycloalkyl optionally substituted with 1-6 R^{1a} or R^{1b} groups, 3-11 membered heterocyclyl optionally substituted with 1-6 R^{1a} or R^{1b} groups, —(CR^{1a}R^{1b})₁₋₅, —(CR^{1a}═CR^{1b})—, —(CR^{1a}R^{1b})₁₋₅-A- wherein A is O, S, or NR^{1c}, —(CR^{1a}R^{1b})₁₋₅-A-(CR^{1a}R^{1b})₁₋₅— wherein A is O, S, or NR^{1c}, —(CR^{1a}R^{1b})₁₋₅-A-(CR^{1a}R^{1b})₁₋₅-A- wherein A is O, S, or NR^{1c}, —(CR^{1a}R^{1b})₁₋₅—(CR^{1a}═CR^{1b})—(CR^{1a}R^{1b})₁₋₅—, —(CR^{1a}R^{1b})₁₋₅—(CR^{1a}═CR^{1b})—(CR^{1a}R^{1b})₁₋₅-A- wherein A is O, S, or NR^{1c}, —(CR^{1a}R^{1b})₁₋₅—(C≡C)—(CR^{1a}R^{1b})₁₋₅—, —(CR^{1a}R^{1b})₁₋₅—(C≡C)—(CR^{1a}R^{1b})₁₋₅-A- wherein A is O, S, or NR^{1c}, —(C≡C)—(CR^{1a}R^{1b})₁₋₅-A-(CR^{1a}R^{1b})₁₋₅— wherein A is O, S, or NR^{1c}, —(C≡C)—(CR^{1a}R^{1b})₁₋₅, —(CR^{1a}R^{1b})₁₋₅-(3-11 membered cycloalkyl optionally substituted with 1-6 R^{1a} or R^{1b} groups)-, —(CR^{1a}R^{1b})₁₋₅-(3-11 membered heterocyclyl optionally substituted with 1-6 R^{1a} or R^{1b} groups)-, -(3-11 membered cycloalkyl optionally substituted with 1-6 R^{1a} or R^{1b} groups)-(CR^{1a}R^{1b})₁₋₅—, -(3-11 membered heterocyclyl optionally substituted with 1-6 R^{1a} or R^{1b} groups)-(CR^{1a}R^{1b})₁₋₅—, —(CR^{1a}R^{1b})₁₋₅-(3-11 membered cycloalkyl optionally substituted with 1-6 R^{1a} or R^{1b} groups)-A-, —(CR^{1a}R^{1b})₁₋₅-(3-11 membered heterocyclyl optionally substituted with 1-6 R^{1a} or R^{1b} groups)-A-, —(CR^{1a}R^{1b})₁₋₅-(3-11 membered cycloalkyl optionally substituted with 1-6 R^{1a} or R^{1b} groups)-(CR^{1a}R^{1b})₁₋₅, —(CR^{1a}R^{1b})₁₋₅-(3-11 membered cycloalkyl optionally substituted with 1-6 R^{1a} or R^{1b} groups)-A- wherein A is O, S, or NR^{1c}, —(CR^{1a}R^{1b})₁₋₅-(3-11 membered cycloalkyl optionally substituted with 1-6 R^{1a} or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 1-6 R^{1a} or R^{1b} groups)- wherein A is O, S, or NR^{1c}, —(CR^{1a}R^{1b})₁₋₅-(3-11 membered heterocyclyl optionally substituted with 1-6 R^{1a} or R^{1b} groups)-(CR^{1a}R^{1b})₁₋₅, —(CR^{1a}R^{1b})₁₋₅-(3-11 membered heterocyclyl optionally substituted with 1-6 R^{1a} or R^{1b} groups)-(CR^{1a}R^{1b})₁₋₅-A- wherein A is O, S, or NR^{1c}, —(CR^{1a}R^{1b})₁₋₅-(3-11 membered heterocyclyl optionally substituted with 1-6 R^{1a} or R^{1b} groups)-A- wherein A is O, S, or NR^{1c}, —(CR^{1a}R^{1b})₁₋₅-A-(3-11 membered heterocyclyl optionally substituted with 1-6 R^{1a} or R^{1b} groups)- wherein A is O, S, or NR^{1c}, —(CR^{1a}R^{1b})₁₋₅-(3-11 membered cycloalkyl optionally substituted with 1-6 R^{1a} or R^{1b} groups)-(CR^{1a}R^{1b})₁₋₅-A- wherein A is O, S, or NR^{1c}, —(CR^{1a}R^{1b})₁₋₅-A-(3-11 membered cycloalkyl optionally substituted with 1-6 R^{1a} or R^{1b} groups)- wherein A is O, S, or NR^{1c}, —(CR^{1a}R^{1b})₁₋₅-A-(3-11 membered cycloalkyl optionally substituted with 1-6 R^{1a} or R^{1b} groups)-(CR^{1a}R^{1b})₁₋₅-A- wherein each A is independently O, S, or NR^{1c}, —(CR^{1a}R^{1b})₁₋₅-A-(3-11 membered heterocyclyl optionally substituted with 1-6 R^{1a} or R^{1b} groups)-(CR^{1a}R^{1b})₁₋₅-A- wherein each A is independently O, S, or NR^{1c}, —(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CR^{1a}R^{1b})₁₋₅-A-(CR^{1a}R^{1b})₁₋₅-A-(CR^{1a}R^{1b})₁₋₅-A- wherein A is O, S, or NR^{1c}, —(CR^{1a}R^{1b})₁₋₅-A-(CO) wherein A is O, S, or NR^{1c}, —(CR^{1a}R^{1b})₁₋₅—(CR^{1a}═CR^{1b})—(CR^{1a}R^{1b})₁₋₅-A-(CO)— wherein A is O, S, or NR^{1c}, —(CR^{1a}R^{1b})₁₋₅—(C≡C)—(CR^{1a}R^{1b})₁₋₅-A-(CO)— wherein A is O, S, or NR^{1c}, —(CR^{1a}R^{1b})₁₋₅-(3-11 membered cycloalkyl optionally substituted with 1-6 R^{1a} or R^{1b} groups)-(CR^{1a}R^{1b})₁₋₅-A-(CO)— wherein A is O, S, or NR^{1c}, —(CR^{1a}R^{1b})₁₋₅-A-(CO)-(3-11 membered cycloalkyl optionally substituted with 1-6 R^{1a} or R^{1b} groups)- wherein A is O, S, or NR^{1c}, —(CR^{1a}R^{1b})₁₋₅-(3-11 membered heterocyclyl optionally substituted with 1-6 R^{1a} or R^{1b} groups)-(CR^{1a}R^{1b})₁₋₅-A-(CO)— wherein A is O, S, or NR^{1c}, —(CR^{1a}R^{1b})₁₋₅-A-(CO)-(3-11 membered heterocyclyl optionally substituted with 1-6 R^{1a} or R^{1b} groups)- wherein A is O, S, or NR^{1c}, —(CR^{1a}R^{1b})₁₋₅-A-(3-11 membered cycloalkyl optionally substituted with 1-6 R^{1a} or R^{1b} groups)-A-(CO)— wherein each A is independently O, S, or NR^{1c}, -(3-11 membered cycloalkyl optionally substituted with 1-6 R^{1a} or R^{1b} groups)-CO—(CR^{1a}R^{1b})₁₋₅-A- wherein A is O, S, or NR^{1c}, —(CR^{1a}R^{1b})₁₋₅-(3-11 membered cycloalkyl optionally substituted with 1-6 R^{1a} or R^{1b} groups)-(CR^{1a}R^{1b})₁₋₅-A-(CO)— wherein A is O, S, or NR^{1c}, —(CR^{1a}R^{1b})₁₋₅-(3-11 membered heterocyclyl optionally substituted with 1-6 R^{1a} or R^{1b} groups)-(CR^{1a}R^{1b})₁₋₅-A-(CO)— wherein A is O, S, or NR^{1c}, -(3-11 membered cycloalkyl optionally substituted with 1-6 R^{1a} or R^{1b} groups)-(CR^{1a}R^{1b})₁₋₅—, or -(3-11 membered heterocyclyl optionally substituted with 1-6 R^{1a} or R^{1b} groups)-(CR^{1a}R^{1b})₁₋₅.

In some embodiments, L in Formula I is —CR^{1a}═CR^{1b}—, such as, for example, —CH═CH—.

In some embodiments, L in Formula I is —(CR^{1a}R^{1b})₁₋₅, for example —(CH₂)₁₋₅—, —CH₂—, —CH₂CH₂CH₂— and the like.

In some embodiments, L in Formula I is —(CR^{1a}R^{1b})₁₋₅-A- wherein A is O, S, or NR^{1c}, such as for example, —(CH₂)₁₋₅—O—, —(CH₂)₁₋₅—S—, —(CH₂)₁₋₅—NH—, or —(CH₂)₀₋₂—(C(CH₃)₂)—(CH₂)₀₋₂—O—.

In other embodiments, L in Formula I is —(CR^{1a}R^{1b})₁₋₅-A-(CR^{1a}R^{1b})₁₋₅— wherein A is O, S, or NR^{1c}, such as, for example, —(CH₂)₁₋₅—O—(CH₂)₁₋₅—, —(CH₂)₁₋₅—S—(CH₂)₁₋₅—, —(CH₂)₁₋₅—NH—(CH₂)₁₋₅—.

In some embodiments, L in Formula I is —(C≡C)—(CR^{1a}R^{1b})₁₋₅, such as, for example, —(C≡C)—(CH₂)₂—, and the like.

In some embodiments, L in Formula I is —(CR^{1a}R^{1b})₁₋₅-(3-11 membered cycloalkyl optionally substituted with 1-6 R^{1a} or R^{1b} groups)-, such as, for example, —CH₂-cyclobutyl-.

In some embodiments, L in Formula I is —(CR^{1a}R^{1b})₁₋₅-(3-11 membered cycloalkyl optionally substituted with 1-6 R^{1a} or R^{1b} groups)-(CR^{1a}R^{1b})₁₋₅, such as, for example, —CH₂— cyclobutyl-CH₂— and the like.

In some embodiments, L in Formula I is —(CR^{1a}R^{1b})₁₋₅-(3-11 membered heterocyclyl optionally substituted with 1-6 R^{1a} or R^{1b} groups)-(CR^{1a}R^{1b})₁₋₅, such as, for example, —CH₂— azetidinyl-CH₂—.

In some embodiments, L in Formula I is —(CR^{1a}R^{1b})₁₋₅-(3-11 membered heterocyclyl optionally substituted with 1-6 R^{1a} or R^{1b} groups)-, such as, for example, —CH₂-azetidinyl-.

In some embodiments, L in Formula I is -(3-11 membered heterocyclyl optionally substituted with 1-6 R^{1a} or R^{1b} groups)-(CR^{1a}R^{1b})₁₋₅—, such as, for example, -azetidinyl-CH₂—, -pyrolidnyl-CH₂—, -piperidinyl-CH₂—, and the like.

In some embodiments, L in Formula I is —(CR^{1a}R^{1b})₁₋₅-(3-11 membered cycloalkyl optionally substituted with 1-6 R^{1a} or R^{1b} groups)-(CR^{1a}R^{1b})₁₋₅-A- wherein A is O, S, or NR^{1c}, such as, for example, —CH₂-cyclopropyl-CH₂—O—, and the like.

In some embodiments, L in Formula I is —(CR^{1a}R^{1b})₁₋₅-(3-11 membered heterocyclyl optionally substituted with 1-6 R^{1a} or R^{1b} groups)-(CR^{1a}R^{1b})₁₋₅-A- wherein A is O, S, or NR^{1c}, such as, for example, —CH₂-piperidinyl-CH₂CH₂—O—, and the like.

In some embodiments, L in Formula I is —(CR^{1a}R^{1b})₁₋₅-(3-11 membered heterocyclyl optionally substituted with 1-6 R^{1a} or R^{1b} groups)-A- wherein A is O, S, or NR^{1c}, such as, for example, —CH₂-azetidinyl-O—, and the like.

In some embodiments, L in Formula I is —(CR^{1a}R^{1b})₁₋₅-A-(3-11 membered heterocyclyl optionally substituted with 1-6 R^{1a} or R^{1b} groups)- wherein A is O, S, or NR^{1c}, such as, for example, —CH₂—O-azetidinyl-, —CH₂—NH-azetidinyl-, and the like.

In other embodiments, L in Formula I is —(CR^{1a}R^{1b})₁₋₅-A-(3-11 membered cycloalkyl optionally substituted with 1-6 R^{1a} or R^{1b} groups)- wherein A is O, S, or NR^{1c}, such as —CH₂—O-cyclobutylene-, —CH₂—NH-cyclobutylene-, and the like.

In some embodiments, L in Formula I is —(CR^{1a}R^{1b})₁₋₅-A-(CR^{1a}R^{1b})₁₋₅-A- wherein A is O, S, or NR^{1c}, such as, for example, —CH₂—O—CH₂CH₂—O—.

In some embodiments, L in Formula I is

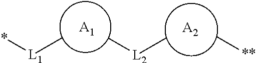

wherein
* is the point of attachment to the N atom to which L attaches in Formula I; and ** is a point of attachment to ULM;
L₁ is a bond, (C(R¹⁰)₂)ₚ or CO;
L₂ is a bond, (C(R¹⁰)₂)ₚ or CO;
each p is independently 1, 2, 3, or 4;
each R¹⁰ is independently H, D, or C₁-C₄ alkyl;
ring A₁ and ring A₂ are each independently 3-11 membered cycloalkyl, optionally substituted with 1-8 R^{1a} or R^{1b} groups, 3-11 membered heterocyclyl optionally substituted with 1-8 R^{1a} or R^{1b} groups, aryl optionally substituted with 1-8 R^{1a} or R^{1b} groups, or heteroaryl optionally substituted with 1-8 R^{1a} or R^{1b} groups;
wherein R^{1a} and R^{1b} are each independently, —H, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C—C≡C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)-CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 1-4 R^{1e} groups.

According to the disclosure, L₁ is a bond, (C(R¹⁰)₂)ₚ or CO. In some embodiments, L₁ is a bond. In embodiments in which L₁ is a bond, it is to be understood that the bond is a chemical bond between A₁ and the N atom to which L attaches in Formula I. In some embodiments, L₁ is (C(R¹⁰)₂)ₚ. In other embodiments, L₁ is CO.

According to the disclosure, L₂ is a bond, (C(R¹⁰)₂)ₚ or CO. In some embodiments, L₂ is a bond. In embodiments in which L₂ is a bond, it is to be understood that the bond is a chemical bond between A₁ and A₂. In some embodiments, L₂ is (C(R¹⁰)₂)ₚ. In other embodiments, L₂ is CO.

According to the disclosure, each p is independently 1, 2, 3, or 4. In some embodiments, each p is 1. In some embodiments, at least one p is 1. In some embodiments, each p is 2. In some embodiments, at least one p is 2. In other embodiments, each p is 3. In other embodiments, at least one p is 3. In other embodiments, each p is 4. In other embodiments, at least one p is 4.

According to the disclosure, each R¹⁰ is independently H, D, or C₁-C₄ alkyl. In some embodiments, each R¹⁰ is H. In some embodiments, at least one R¹⁰ is H. In some embodiments, each R¹⁰ is D. In some embodiments, at least one R¹⁰ is D. In other embodiments, each R¹⁰ is C₁-C₄ alkyl. In other embodiments, at least one R¹⁰ is C₁-C₄ alkyl. In other embodiments, each R¹⁰ is methyl or ethyl. In other embodiments, at least one R¹⁰ is methyl or ethyl.

According to the disclosure, ring A₁ is a 3-7 membered cycloalkyl group, a 4-10-membered heterocycloalkyl group, an aryl group, or a heteroaryl group. In some embodiments, ring A₁ is a 3-7 membered cycloalkyl group. In some embodiments, ring A₁ is a 4-10-membered heterocycloalkyl group. In other embodiments, ring A₁ is an aryl group. In other embodiments, ring A₁ is a heteroaryl group.

In some embodiments, ring A₁ is a piperazine group, a morpholine group, a piperidine group, a pyrrolidine group, an azetidine group or an azabicyclo-alkyl group. In other embodiments, ring A₁ is a piperidine or pyrrolidine group.

According to the disclosure, ring A₂ is a 3-7 membered cycloalkyl group, a 4-10-membered heterocycloalkyl group, an aryl group, or a heteroaryl group. In some embodiments, ring A₂ is a 3-7 membered cycloalkyl group. In some embodiments, ring A₂ is a 4-10-membered heterocycloalkyl group. In other embodiments, ring A₂ is an aryl group. In other embodiments, ring A₂ is a heteroaryl group.

In some embodiments, ring A₂ is a piperazine group, a morpholine group, a piperidine group, a pyrrolidine group, an azetidine group, a diazaspiroalkyl group or an azabicycloalkyl group. In other embodiments, ring A₂ is a piperazine group or a diazaspirononane group.

In some embodiments, L in Formula I is

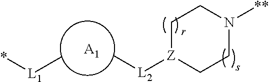

wherein
L₁, L₂ and ring A₁ are as defined herein;
r is 0, 1 or 2;
s is 0, 1 or 2; and
Z is N or CR¹⁰; and
R¹⁰ is as defined herein.

According to the disclosure, r is 0, 1 or 2. In some embodiments, r is 0. In some embodiments, r is 1. In other embodiments, r is 2.

According to the disclosure, s is 0, 1 or 2. In some embodiments, s is 0. In some embodiments, s is 1. In other embodiments, s is 2.

According to the disclosure, Z is N or CR¹⁰. In some embodiments, Z is N. In other embodiments, Z is CR¹⁰.

In some embodiments, the compounds of Formula (I) are represented by compounds of Formula II

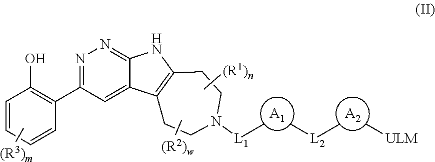

or a pharmaceutically acceptable salt thereof; wherein each ULM, (R¹)ₙ, (R²)_{w}, (R³)ₘ, L₁, L₂, ring A₁ and ring A₂ are defined with respect to Formula (I).

In some aspects of the disclosure, ULM in Formula I is

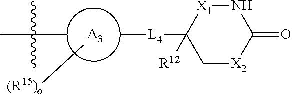

wherein:
[symbol] is a point of attachment to L;
Ring A₃ is a monocyclic, bicyclic or tricyclic aryl, heteroaryl or heterocycle group,
L₄ is a bond, —O—, —S—, —NR^{a}—, —C(R^{a})₂— —C(O)NR^{a}—;
X₁ is CH₂, CO, CH═CH (when X₂═CO), or N═CH (when X₂═CO);
X₂ is CH₂, CO, CH═CH (when X₁═CO), or N═CH (when X₁═CO);
R¹² is H, D, optionally substituted C₁₋₄ alkyl, C₁₋₄ alkoxy, C₁₋₄haloalkyl, —CN, —OR^{a}, —OR^{b} or —SR^{b};
each R¹⁵ is independently H, D, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}; and
o is 1, 2, 3, 4, or 5.

According to the disclosure, Ring A₃ is a monocyclic, bicyclic or tricyclic aryl, heteroaryl or heterocycle group. In some embodiments, Ring A₃ is a monocyclic, bicyclic or tricyclic aryl group. In other embodiments, Ring A₃ is a monocyclic, bicyclic or tricyclic heteroaryl group. In other embodiments, Ring A₃ is a monocyclic, bicyclic or tricyclic heterocycle group.

In some embodiments, Ring A₃ is a bicyclic heterocycle group. In some embodiments, Ring A₃ is an isoindoline group. In other embodiments, Ring A₃ is an isoindolin-1-one group. In other embodiments, Ring A₃ is an isoindolin-3-one group. In other embodiments, Ring A₃ is an isoindoline-1,3-dione group.

According to the disclosure, each R¹⁵ is independently H, D, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

In some embodiments, each R¹⁵ is H. In some embodiments, at least one R¹⁵ is H. In some embodiments, each R¹⁵ is D. In some embodiments, at least one R¹⁵ is D. In some embodiments, each R¹⁵ is C₁-C₆alkyl. In some embodiments, at least one R¹⁵ is C₁-C₆alkyl. In some embodiments, each R¹⁵ is methyl or ethyl. In some embodiments, at least one R¹⁵ is methyl or ethyl.

In other embodiments, each R¹⁵ is independently selected from halogen, oxo, —OH, —CN, —NO₂, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

According to the disclosure, o is 1, 2, 3, 4, or 5. In some embodiments, o is 1. In some embodiments, o is 2. In other embodiments, o is 3. In other embodiments, o is 4. In other embodiments, o is 5.

According to the disclosure, L₄ is a bond, —O—, —S—, —NR^{a}—, —C(R^{a})₂— —C(O)NR^{a}—. In some embodiments, L₄ is a bond. In some embodiments, L₄ is —O—. In other embodiments, L₄ is a —S—. In other embodiments, L₄ is —NR^{a}—. In other embodiments, L₄ is —C(R^{a})₂—. In other embodiments, L₄ is —C(O)NR^{a}—.

According to the disclosure, X₁ is CH₂, CO, CH═CH (when X₂═CO), or N═CH (when X₂═CO). In some embodiments, X₁ is CH₂. In some embodiments, X₁ is CO. In other embodiments, X₁ is CH═CH (when X₂═CO). In other embodiments, X₁ is N═CH (when X₂═CO).

According to the disclosure, X₂ is CH₂, CO, CH═CH (when X₁═CO), or N═CH (when X₁═CO). In some embodiments, X₂ is CH₂. In some embodiments, X₂ is CO. In other embodiments, X₂ is CH═CH (when X₁═CO). In other embodiments, X₂ is N═CH (when X₁═CO).

According to the disclosure, R¹² is H, D, optionally substituted C₁₋₄ alkyl, C₁₋₄ alkoxy, C₁₋₄ haloalkyl, —CN, —OR^{a}, —OR^{b} or —SR^{b}. In some embodiments, R¹² is H. In some embodiments, R¹² is D. In some embodiments, R¹² is optionally substituted C₁₋₄ alkyl. In other embodiments, R¹² is C₁₋₄ alkoxy. In other embodiments, R¹² is C₁₋₄haloalkyl. In other embodiments, R¹² is —CN. In other embodiments, R¹² is —OR^{a}. In other embodiments, R¹² is —OR^{b}. In other embodiments, R¹² is —SR^{b}.

In some embodiments of ULM, Ring A₃ is a bicyclic or tricyclic heteroaryl or heterocycloalkyl group. In some embodiments of ULM, Ring A₃ is heteroaryl bicyclic. In some embodiments of ULM, Ring A₃ is heteroaryl tricyclic. In some embodiments of ULM, Ring A₃ is heterobicycloalkyl. In some embodiments of ULM, Ring A₃ is heterotricycloalkyl.

In other embodiments of ULM, Ring A₃ is a monocyclic heteroaryl having at least one N atom. In other embodiments of ULM, Ring A₃ is a pyridine or a pyridazine. In other embodiments of ULM, Ring A₃ is

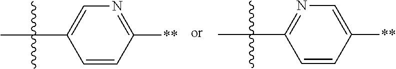

wherein [symbol] is a point of attachment to L and ** is a point of attachment to L₄.

In yet other embodiments, Ring A₃ is

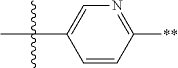

wherein [symbol] is a point of attachment to L and ** is a point of attachment to L₄. In yet other embodiments, Ring A₃ is

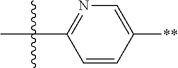

wherein [symbol] is a point of attachment to L and ** is a point of attachment to L₄.

In other embodiments of ULM, Ring A₃ is a bicyclic heteroaryl having at least one N atom. In other embodiments of ULM, Ring A₃ is an isoindolin-one, an isoindolin-dione, an isoquinolin-one or an isoquinolin-dione. In other embodiments of ULM, Ring A₃ is

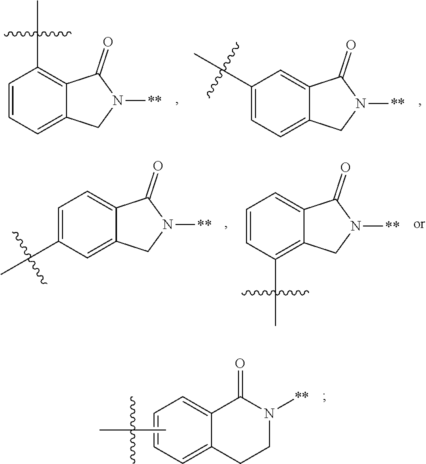

wherein [symbol] is a point of attachment to L and ** is a point of attachment to L₄.

In yet other embodiments, Ring A₃ is

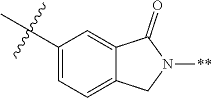

wherein [symbol] is a point of attachment to L and ** is a point of attachment to L₄. In yet other embodiments, Ring A₃ is

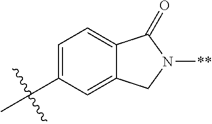

wherein [symbol] is a point of attachment to L and ** is a point of attachment to L₄.

In yet other embodiments of ULM, Ring A₃ is

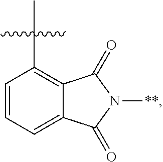

wherein [symbol] is a point of attachment to L and ** is a point of attachment to L₄. In yet other embodiments of ULM, Ring A₃ is

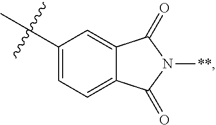

wherein [symbol] is a point of attachment to L and ** is a point of attachment to L₄.

In yet other embodiments of ULM, Ring A₃ is

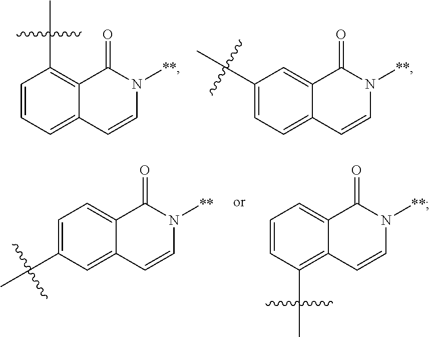

wherein [symbol] is a point of attachment to L and ** is a point of attachment to L₄.

In yet other embodiments of ULM, Ring A₃ is a tricyclic heteroaryl having at least one N atom. In yet other embodiments of ULM, Ring A₃ is a carbazole, a pyrido-indole or a pyrrolo-dipyridine. In yet other embodiments of ULM, Ring A₃ is

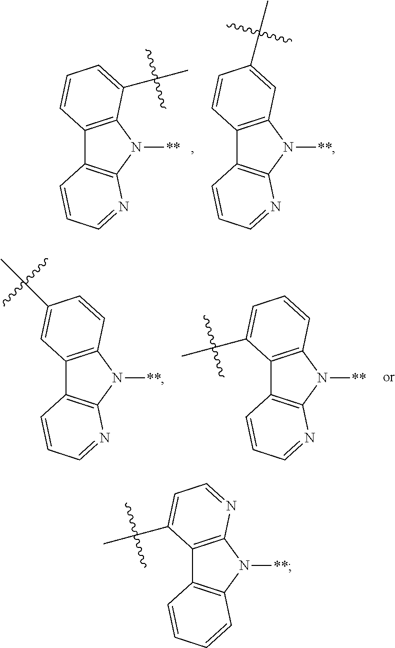

wherein [symbol] is a point of attachment to L and ** is a point of attachment to L₄.

In yet other embodiments of ULM, Ring A₃ is

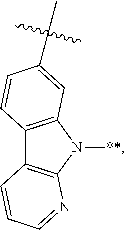

wherein [symbol] is a point of attachment to L and ** is a point of attachment to L₄. In yet other embodiments of ULM, Ring A₃ is

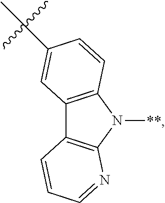

wherein [symbol] is a point of attachment to L and ** is a point of attachment to L₄.

According to the disclosure, ULM in Formula I is

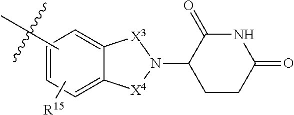

wherein:
[symbol] is a point of attachment to L;
R¹⁵ is as defined herein;
X₃ is CH₂, CO, CH═CH (when X₄═CO), or N═CH (when X₄═CO); and
X₄ is CH₂, CO, CH═CH (when X₃═CO), or N═CH (when X₃═CO).

According to the disclosure, X₃ is CH₂, CO, CH═CH (when X₄═CO), or N═CH (when X₄═CO). In some embodiments, X₃ is CH₂. In some embodiments, X₃ is CO. In other embodiments, X₃ is CH═CH (when X₄═CO). In other embodiments, X₃ is N═CH (when X₄═CO).

According to the disclosure, X₄ is CH₂, CO, CH═CH (when X₃═CO), or N═CH (when X₃═CO). In some embodiments, X₄ is CH₂. In some embodiments, X₄ is CO. In other embodiments, X₄ is CH═CH (when X₃═CO). In other embodiments, X₄ is N═CH (when X₃═CO).

According to the disclosure, ULM in Formula I is a moiety having the Formula B-I

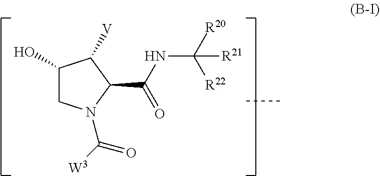

wherein
the dashed line ( [symbol] ) indicates the position of attachment of B-I to L;
V is H or F;
R²⁰ is optionally substituted phenyl, optionally substituted napthyl, or an optionally substituted 5-10 membered heteroaryl;
one of R²¹ or R²² is H, D, haloalkyl, optionally substituted alkyl, optionally substituted cycloalkyl, optionally substituted heterocycloalkyl, —COR^{g}, CONR^{h1}R^{h2}; the other of R²¹ or R²² is H or D;
or R²¹ and R²², together with the carbon atom to which they are both attached, form an optionally substituted 3-5 membered cycloalkyl, heterocyclyl;
W³ is an optionally substituted aryl, optionally substituted heteroaryl, or

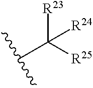

R²³ and R²⁴ are independently H, D, optionally substituted alkyl, optionally substituted cycloalkyl, or optionally substituted haloalkyl,
or R²³, R²⁴, and the carbon atom to which they are attached form an optionally substituted cycloalkyl or optionally substituted heterocyclyl;
R²⁵ is an optionally substituted heterocyclyl, optionally substituted heteroaryl, optionally substituted aryl, CONRⁱR^{j}, NRⁱR^{j},

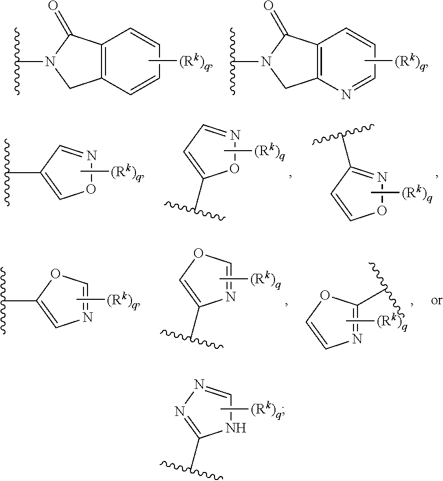

Rⁱ is selected from H or optionally substituted alkyl;
R^{j} is selected from H, —C(O)—* wherein * is a point of attachment to L, optionally substituted alkyl, optionally substituted alkylcarbonyl, optionally substituted (cycloalkyl)alkylcarbonyl, optionally substituted aralkylcarbonyl, optionally substituted arylcarbonyl, optionally substituted (cycloalkyl)carbonyl, optionally substituted (heterocyclyl) carbonyl, or optionally substituted aralkyl;
each R^{k} is independently H, halo, optionally substituted alkoxy, cyano, optionally substituted alkyl, haloalkyl, or haloalkoxy;
each R^{g} is independently selected from H, optionally substituted alkyl or NR^{h1}R^{h2};
each R^{h1} and R^{h2} is independently H, D, optionally substituted alkyl, or R^{h1} and R^{h2} together with the nitrogen atom to which they are attached form a 4-7 membered heterocyclyl; and
q is 0, 1, 2, 3, or 4.

In some embodiments of B-I, V is H.

In other embodiments of B-I, V is F.

In some embodiments of B-I, R²⁰ is optionally substituted phenyl having the formula:

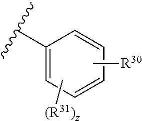

wherein
R³⁰ is H, D, halo, —CN, —OH, —NO₂, —NR^{h1}R^{h2}, —OR^{h1}, —CONR^{h1}R^{h2}, —NR^{h1}COR^{h2}, —SO₂NR^{h1}R^{h2}, —NR^{h1}SO₂R^{h2}, optionally substituted alkyl, optionally substituted alkoxy, optionally substituted haloalkyl, optionally substituted haloalkoxy; optionally substituted aryl; optionally substituted heteroaryl; optionally substituted cycloalkyl; or optionally substituted heterocyclyl;
R³¹ is H, D, halo, CN, optionally substituted alkyl, optionally substituted haloalkyl, hydroxy, or optionally substituted haloalkoxy; and
z is 0, 1, 2, 3, or 4.

In some embodiments of B-I, R²⁰ is optionally substituted phenyl, R³⁰ is an optionally substituted heteroaryl.

In some embodiments of B-I, R²⁰ is optionally substituted phenyl, R³⁰ is

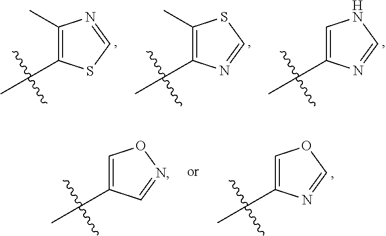

each optionally substituted.

In other embodiments of B-I, R²⁰ is optionally substituted phenyl, R³⁰ is

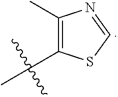 [SMILES: *c1scnc1C]

In other embodiments, R²⁰ is

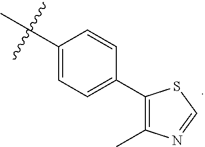 [SMILES: *c1ccc(-c2scnc2C)cc1]

In some embodiments of B-I, R²⁰ is optionally substituted phenyl, R³¹ is hydroxy, halogen, —NH(C₁-C₄alkyl), or C₁-C₆alkoxy, and z is 0, 1, 2, 3, or 4.

In some embodiments of B-I, one of R²¹ or R²² is H, and the other of R²¹ or R²² is H or optionally substituted alkyl.

In other embodiments of B-I-I, one of R²¹ or R²² is H, and the other of R²¹ or R²² is optionally substituted C₁-C₆alkyl.

In other embodiments of B-I, one of R²¹ or R²² is H, and the other of R²¹ or R²² is C₁-C₆alkyl.

In other embodiments of B-I, one of R²¹ or R²² is H, and the other of R²¹ or R²² is —CH₃.

In other embodiments of B-I, both R²¹ and R²² are H.

In some embodiments of B-I, W³ is

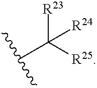

In some embodiments of B-I, R²³ is H.

In some embodiments of B-I, R²⁴ is H, or optionally substituted alkyl.

In some embodiments of B-I, R²⁴ is H.

In some embodiments of B-I, R²⁴ is optionally substituted alkyl.

In some embodiments of B-I, R²⁴ is optionally substituted C₁-C₆alkyl.

In some embodiments of B-I, R²⁴ is C₁-C₆alkyl.

In some embodiments of B-I, R²⁴ is C₁-C₆alk-OH, C₁-C₆alk-NH₂, —C₁-C₆alk-CONH—*, or —C₁-C₆alk-NHCO—* wherein * is a point of attachment to L.

In some embodiments of B-I, R²⁴ is -t-butyl or -isopropyl.

In some embodiments of B-I, R²¹ is NR^{a}R^{b}.

In some embodiments of B-I, Rⁱ is H or optionally substituted alkyl.

In some embodiments of B-I, Rⁱ is H.

In some embodiments of B-I, R^{j} is H, optionally substituted alkyl, —C(O)—* wherein * is a point of attachment to L, optionally substituted (cycloalkyl)carbonyl, or optionally substituted alkylcarbonyl.

In some embodiments of B-I, R^{j} is optionally substituted alkylcarbonyl.

In some embodiments of B-I, R^{j} is —C(O)—* wherein * is a point of attachment to L.

In some embodiments of B-I, R²⁵ is CONRⁱR^{j}.

In some embodiments of B-I, R²⁵ is

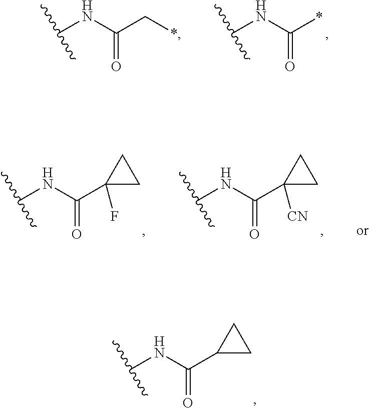

wherein * is a point of attachment to L.

In some embodiments of B-I, R²⁵ is

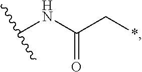

wherein * is a point of attachment to R¹.

In some embodiments of B-I, R²⁵ is

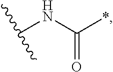

wherein * is a point of attachment to R¹.

In some embodiments of B-I, R²⁵ is

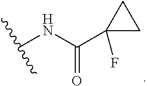 [SMILES: *NC(=O)C1(F)CC1]

In some embodiments of B-I, R²⁵ is —NH—* wherein * is a point of attachment to R¹.

In some embodiments of B-I, R²⁵ is optionally substituted heteroaryl.

In some embodiments of B-I, R²⁵ is

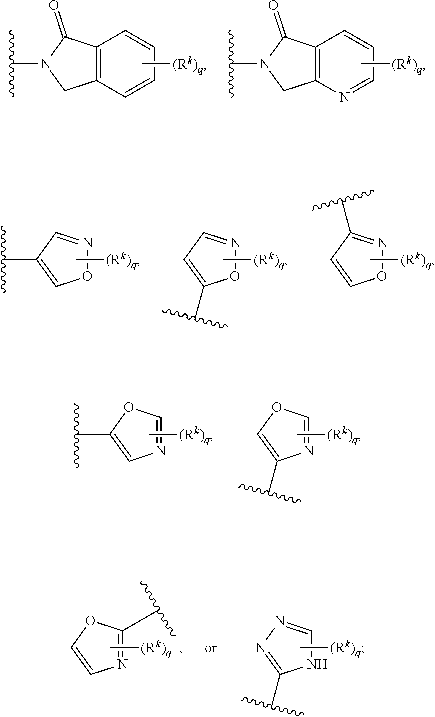

wherein each R^{k} is independently halo, optionally substituted alkoxy, cyano, optionally substituted alkyl, haloalkyl, or haloalkoxy, and q is 0, 1, or 2.

In some embodiments, R²⁵ is

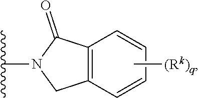

wherein each R^{k} is independently halo, optionally substituted alkoxy, cyano, optionally substituted alkyl, haloalkyl, or haloalkoxy, and q is 0, 1, or 2.

In some embodiments, R²⁵ is

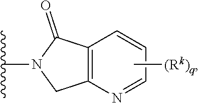

wherein each R^{k} is independently halo, optionally substituted alkoxy, cyano, optionally substituted alkyl, haloalkyl, or haloalkoxy, and q is 0, 1, or 2.

In some embodiments, R²⁵ is

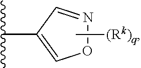

wherein each R^{k} is independently halo, optionally substituted alkoxy, cyano, optionally substituted alkyl, haloalkyl, or haloalkoxy, and q is 0, 1, or 2.

In some embodiments, R²⁵ is

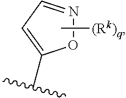

wherein each R^{k} is independently halo, optionally substituted alkoxy, cyano, optionally substituted alkyl, haloalkyl, or haloalkoxy, and q is 0, 1, or 2.

In some embodiments, R²⁵ is

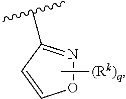

wherein each R^{k} is independently halo, optionally substituted alkoxy, cyano, optionally substituted alkyl, haloalkyl, or haloalkoxy, and q is 0, 1, or 2.

In some embodiments, R²⁵ is

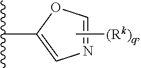

wherein each R^{k} is independently halo, optionally substituted alkoxy, cyano, optionally substituted alkyl, haloalkyl, or haloalkoxy, and q is 0, 1, or 2.

In some embodiments, R²⁵ is

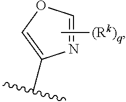

wherein each R^{k} is independently halo, optionally substituted alkoxy, cyano, optionally substituted alkyl, haloalkyl, or haloalkoxy, and q is 0, 1, or 2.

In some embodiments, R²⁵ is

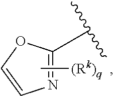

wherein each R^{k} is independently halo, optionally substituted alkoxy, cyano, optionally substituted alkyl, haloalkyl, or haloalkoxy, and q is 0, 1, or 2.

In some embodiments, R²⁵ is

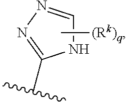

wherein each R^{k} is independently halo, optionally substituted alkoxy, cyano, optionally substituted alkyl, haloalkyl, or haloalkoxy, and q is 0, 1, or 2.

In some embodiments, B-I is a compound of formula:

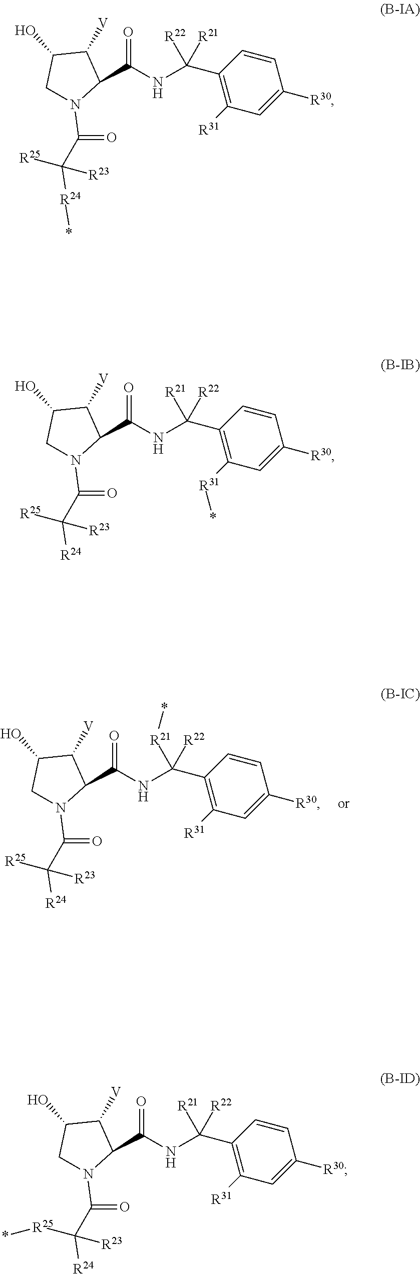

wherein * is a point of attachment to L.

In some embodiments of B-IA, B-IB, B-IC, or B-ID, R³⁰ is optionally substituted

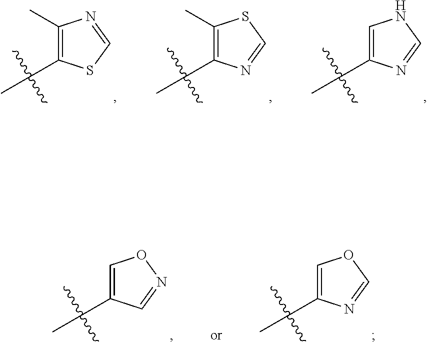

and R³¹ is H, D, hydroxy, halogen, aminoC₁₋₄alkyl, or C₁₋₄alkyloxy.

In some embodiments, the ULM in Formula I is

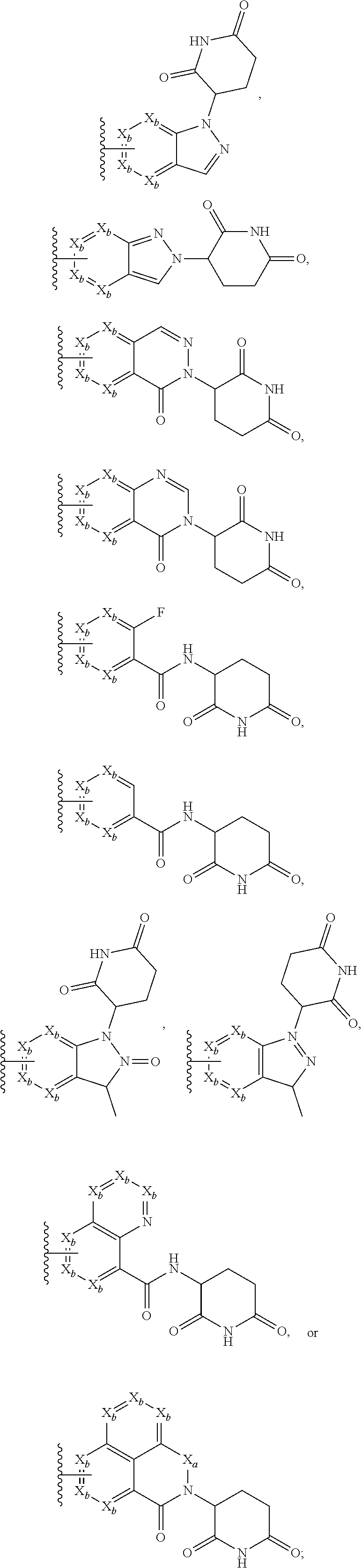

wherein [symbol] is a point of attachment to L;
Xₐ is a bond, —C(O)—, —C(S)—, —CH₂—, —CHCF₃—, SO₂—, —S(O), P(O)R^{b}— or —P(O)OR^{b}—;
R^{b}, is H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl; and
each X_{b} is independently N, or CR^{b}, provided that one X_{b} is a C atom having the attachment point to L.

In some embodiments, the ULM is

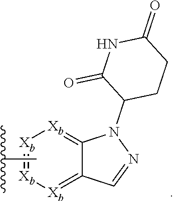

In some embodiments, the ULM is

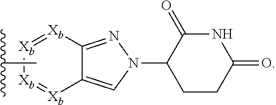

In some embodiments, the ULM is

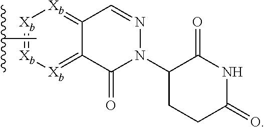

In some embodiments, the ULM is

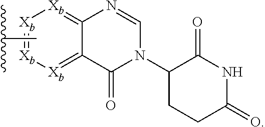

In some embodiments, the ULM is

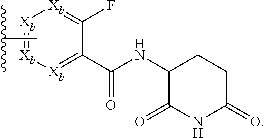

In some embodiments, the ULM is

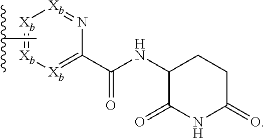

In some embodiments, the ULM is

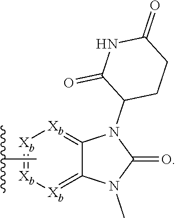

In some embodiments, the ULM is

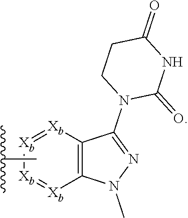

In some embodiments, the ULM is

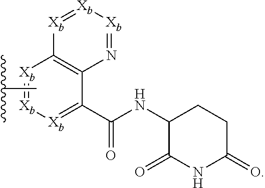

In some embodiments, the ULM is

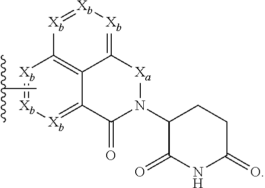

In some embodiments, Xₐ in the ULM is a bond, —C(O)—, —C(S)—, —CH₂—, —CHCF₃—, SO₂—, —S(O), P(O)R^{b}— or —P(O)OR^{b}—.

In some embodiments, Xₐ in the ULM is a bond.

In some embodiments, Xₐ in the ULM is —C(O)—.

In some embodiments, Xₐ in the ULM is —C(S)—.

In some embodiments, Xₐ in the ULM is —CH₂—.

In some embodiments, Xₐ in the ULM is —CHCF₃—.

In some embodiments, Xₐ in the ULM is —SO₂—.

In some embodiments, Xₐ in the ULM is —S(O).

In some embodiments, Xₐ in the ULM is —P(O)R^{b}.

In some embodiments, Xₐ in the ULM is —P(O)OR^{b}—.

In some embodiments, R^{b} in the ULM is H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl.

In some embodiments, R^{b} in the ULM is H.

In some embodiments, R^{b} in the ULM is D.

In some embodiments, R^{b} in the ULM is —C₁-C₆ alkyl.

In some embodiments, R^{b} in the ULM is —C₂-C₆ alkenyl.

In some embodiments, R^{b} in the ULM is —C₂-C₆ alkynyl.

In some embodiments, R^{b} in the ULM is aryl.

In some embodiments, R^{b} in the ULM is cycloalkyl.

In some embodiments, R^{b} in the ULM is cycloalkenyl.

In some embodiments, R^{b} in the ULM is heteroaryl.

In some embodiments, R^{b} in the ULM is heterocycloalkyl.

In some embodiments, R^{b} in the ULM is heterocycloalkenyl.

In some embodiments, each X_{b} in the ULM is independently N, or CR^{b}, provided that one X_{b} is a C atom having the attachment point to PTM.

In some embodiments, each X_{b} in the ULM is CR^{b}, provided that one X_{b} is a C atom having the attachment point to L.

In some embodiments, at least one X_{b} in the ULM is CR^{b}, provided that one X_{b} is a C atom having the attachment point to L.

In some embodiments, at least two X_{b} in the ULM is CR^{b}, provided that one X_{b} is a C atom having the attachment point to L.

In some embodiments, at least three X_{b} in the ULM is CR^{b}, provided that one X_{b} is a C atom having the attachment point to L.

In some embodiments, at least four X_{b} in the ULM is CR^{b}, provided that one X_{b} is a C atom having the attachment point to L.

In some embodiments, at least five X_{b} in the ULM is CR^{b}, provided that one X_{b} is a C atom having the attachment point to L.

In some embodiments, each X_{b} in the ULM is N, provided that one X_{b} is a C atom having the attachment point to L.

In some embodiments, at least one X_{b} in the ULM is N, provided that one X_{b} is a C atom having the attachment point to L.

In some embodiments, at least two X_{b} in the ULM is N, provided that one X_{b} is a C atom having the attachment point to L.

In some embodiments, at least three X_{b} in the ULM is N, provided that one X_{b} is a C atom having the attachment point to L.

In some embodiments, at least four X_{b} in the ULM is N, provided that one X_{b} is a C atom having the attachment point to L.

In some embodiments, at least five X_{b} in the ULM is N, provided that one X_{b} is a C atom having the attachment point to L.

In some embodiments, the compounds of Formula (I) are represented by compounds of Formula III

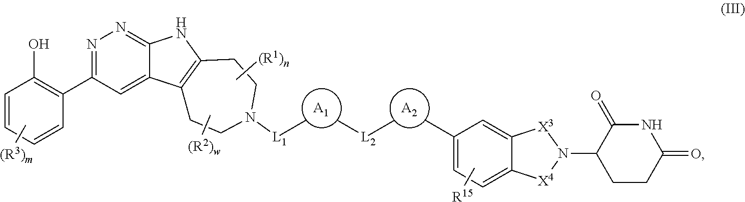

or a pharmaceutically acceptable salt thereof; wherein each (R¹)ₙ, (R²)_{w}, (R³)ₘ, R₁₅, L₁, L₂, ring A₁, ring A₂, X₃, and X₄ are defined with respect to Formula (I).

In some embodiments, the compounds of Formula (I) are represented by compounds of Formula IV

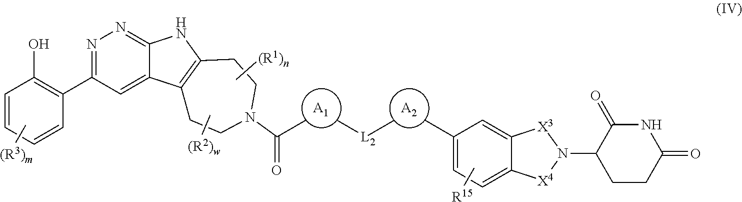

or a pharmaceutically acceptable salt thereof; wherein each (R¹)ₙ, (R²)_{w}, (R³)ₘ, R¹⁵, ring A₁, L₂, ring A₂, X₃ and X₄ are defined with respect to Formula (I).

In some embodiments, the compounds of Formula (I) are represented by compounds of Formula V

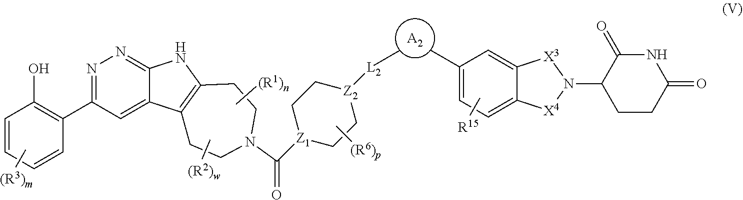

or a pharmaceutically acceptable salt thereof; wherein each (R¹)ₙ, (R²)_{w}, (R³)ₘ, R¹⁵, L₂, ring A₂, X₃ and X₄ are defined with respect to Formula (I); and wherein
Z₁ is N or CR⁶;
Z₂ is N or CR⁶;
each R⁶ is independently H, D, C₁₋₆ alkyl, C₃₋₆ cycloalkyl, or haloalkyl; and
p is 1, 2, 3, 4, 5, 6, 7 or 8.

According to the disclosure, Z₁ is N or CR⁶. In some embodiments, Z₁ is N. In other embodiments, Z₁ is CR⁶.

According to the disclosure, Z₂ is N or CR⁶. In some embodiments, Z₂ is N. In other embodiments, Z₂ is CR⁶.

In some embodiments, Z₁ is N and Z₂ is N. In other embodiments, Z₁ is N and Z₂ is CR⁶.

According to the disclosure, each R⁶ is independently H, D, C₁₋₆ alkyl, C₃₋₆ cycloalkyl, or haloalkyl. In some embodiments, each R⁶ is H. In some embodiments, each R⁶ is D. In other embodiments, each R⁶ is C₁₋₆ alkyl. In other embodiments, each R⁶ is C₃₋₆ cycloalkyl. In other embodiments, each R⁶ is haloalkyl.

In some embodiments, at least one R⁶ is H. In some embodiments, at least one R⁶ is D. In other embodiments, at least one R⁶ is C₁₋₆ alkyl. In other embodiments, at least one R⁶ is C₃₋₆ cycloalkyl. In other embodiments, at least one R⁶ is haloalkyl.

According to the disclosure, p is 1, 2, 3, 4, 5, 6, 7 or 8. In some embodiments, p is 1. In some embodiments, p is 2. In some embodiments, p is 3. In other embodiments, p is 4. In other embodiments, p is 5. In other embodiments, p is 6. In yet other embodiments, p is 7. In yet other embodiments, p is 8.

In some embodiments, the compounds of Formula (I) are represented by compounds of Formula VI

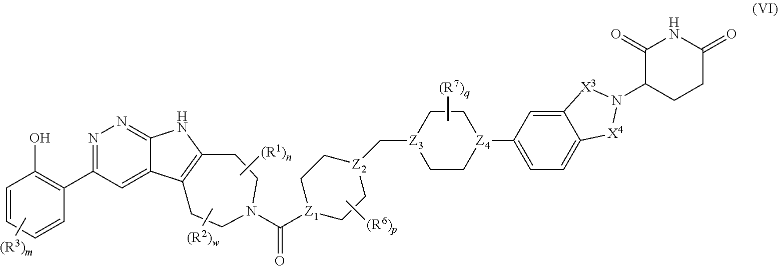

or a pharmaceutically acceptable salt thereof; wherein each (R¹)ₙ, (R²)_{w}, (R³)ₘ, (R⁶)ₚ, Z₁ and Z₂ are defined with respect to Formula I and Formula V; and wherein
Z₃ is N or CR⁶;
Z₄ is N or CR⁶;
each R₇ is independently H, D, C₁₋₆ alkyl, C₃₋₆ cycloalkyl, or haloalkyl; and
q is 1, 2, 3, 4, 5, 6, 7 or 8.

According to the disclosure, Z₃ is N or CR⁶. In some embodiments, Z₃ is N. In other embodiments, Z₃ is CR⁶.

According to the disclosure, Z₄ is N or CR⁶. In some embodiments, Z₄ is N. In other embodiments, Z₄ is CR⁶.

In some embodiments, Z₃ is N and Z₄ is N. In other embodiments, Z₃ is N and Z₄ is CR⁶.

According to the disclosure, each R₇ is independently H, D, C₁₋₆ alkyl, C₃₋₆ cycloalkyl, or haloalkyl. In some embodiments, each R₇ is H. In some embodiments, each R₇ is D. In other embodiments, each R₇ is C₁₋₆ alkyl. In other embodiments, each R₇ is C₃₋₆ cycloalkyl. In other embodiments, each R₇ is haloalkyl.

In some embodiments, at least one R₇ is H. In some embodiments, at least one R₇ is D. In other embodiments, at least one R₇ is C₁₋₆ alkyl. In other embodiments, at least one R₇ is C₃₋₆ cycloalkyl. In other embodiments, at least one R₇ is haloalkyl.

According to the disclosure, q is 1, 2, 3, 4, 5, 6, 7 or 8. In some embodiments, q is 1. In some embodiments, q is 2. In some embodiments, q is 3. In other embodiments, q is 4. In other embodiments, q is 5. In other embodiments, q is 6. In yet other embodiments, q is 7. In yet other embodiments, q is 8.

In some embodiments, the compounds of Formula (I) are represented by compounds of Formula VII

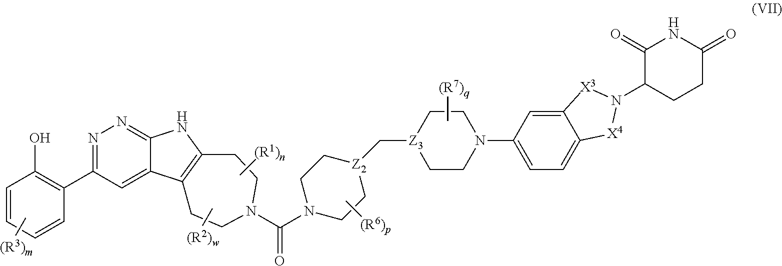

or a pharmaceutically acceptable salt thereof; wherein each (R¹)ₙ, (R²)_{w}, (R³)ₘ, (R⁶)ₚ, (R⁷)_{q}, X³, X⁴, Z₂ and Z₃ are defined with respect to Formula I, Formula V and Formula VI above.

In some embodiments, the compounds of Formula (I) are represented by compounds of Formula VIIIa or formula VIIIb:

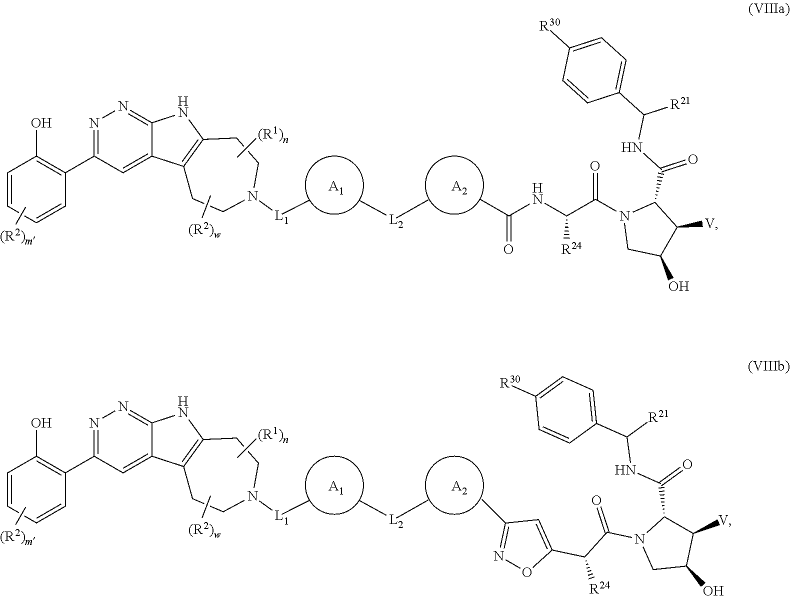

or a pharmaceutically acceptable salt thereof; wherein each (R¹)ₙ, (R²)_{w}, (R³)ₘ, R²¹, R²⁴, R³⁰, V, L₁, L₂, ring A₁ and ring A₂, are defined with respect to Formula (I) or as defined herein.

In some embodiments, the compounds of Formula (I) are represented by compounds of Formula IXa or formula IXb:

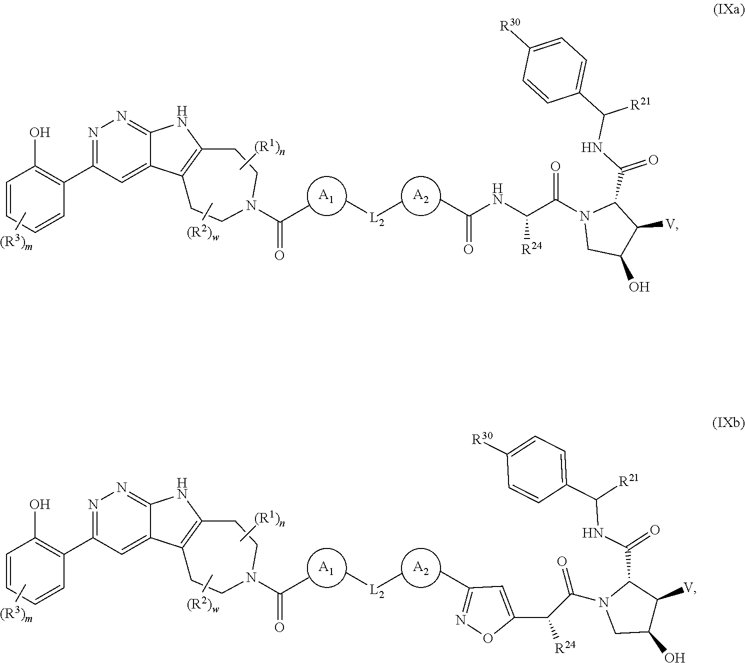

or a pharmaceutically acceptable salt thereof; wherein each (R¹)ₙ, (R²)_{w}, (R³)ₘ, R²¹, R²⁴, R³⁰, V, L₂, ring A₁ and ring A₂, are defined with respect to Formula (I) or as defined herein.

In some embodiments, the compounds of Formula (I) are represented by compounds of Formula Xₐ or formula X_{b}:

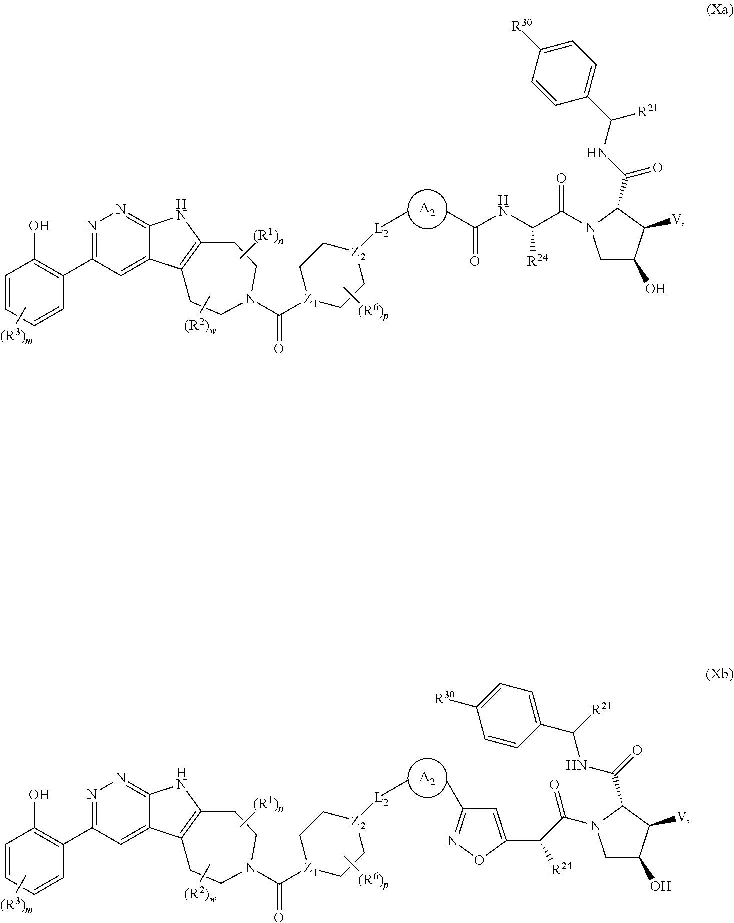

wherein each (R′)ₙ, (R²)_{w}, (R³)ₘ, R²¹, R²⁴, R³⁰, V, L₂, ring A₁ and ring A₂, are defined with respect to Formula (I) or as defined herein; and wherein
Z₁ is N or CR⁶;
Z₂ is N or CR⁶;
each R⁶ is independently H, D, C₁₋₆ alkyl, C₃₋₆ cycloalkyl, or haloalkyl; and
p is 0, 1, 2, 3, 4, 5, 6, 7 or 8.

According to the disclosure, Z₁ is N or CR⁶. In some embodiments, Z₁ is N. In other embodiments, Z₁ is CR⁶.

According to the disclosure, Z₂ is N or CR⁶. In some embodiments, Z₂ is N. In other embodiments, Z₂ is CR⁶.

In some embodiments, Z₁ is N and Z₂ is N. In other embodiments, Z₁ is N and Z₂ is CR⁶.

According to the disclosure, each R⁶ is independently H, D, C₁₋₆ alkyl, C₃₋₆ cycloalkyl, or haloalkyl. In some embodiments, each R⁶ is H. In some embodiments, each R⁶ is D. In other embodiments, each R⁶ is C₁₋₆ alkyl. In other embodiments, each R⁶ is C₃₋₆ cycloalkyl. In other embodiments, each R⁶ is haloalkyl.

In some embodiments, at least one R⁶ is H. In some embodiments, at least one R⁶ is D. In other embodiments, at least one R⁶ is C₁₋₆ alkyl. In other embodiments, at least one R⁶ is C₃₋₆ cycloalkyl. In other embodiments, at least one R⁶ is haloalkyl.

According to the disclosure, p is 1, 2, 3, 4, 5, 6, 7 or 8. In some embodiments, p is 1. In some embodiments, p is 2. In some embodiments, p is 3. In other embodiments, p is 4. In other embodiments, p is 5. In other embodiments, p is 6. In yet other embodiments, p is 7. In yet other embodiments, p is 8.

In some embodiments, the compounds of Formula (I) are represented by compounds of Formula XIa or formula XIb:

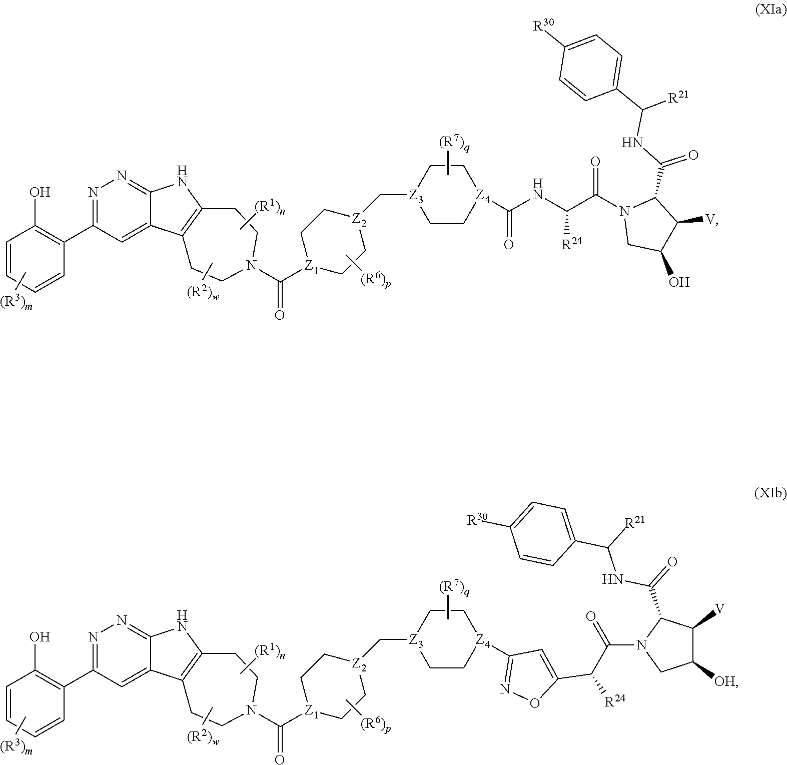

or a pharmaceutically acceptable salt thereof; wherein each (R′)ₙ, (R²)_{w}, (R³)ₘ, (R⁶)ₚ, R²¹, R²⁴, R³⁰, V, Z₁ and Z₂ are defined with respect to Formula I and Formula V; and wherein
Z₃ is N or CR⁷;
Z₄ is N or CR⁷;
each R⁷ is independently H, D, C₁₋₆ alkyl, C₃₋₆ cycloalkyl, or haloalkyl; and
q is 0, 1, 2, 3, 4, 5, 6, 7 or 8.

According to the disclosure, Z₃ is N or CR⁶. In some embodiments, Z₃ is N. In other embodiments, Z₃ is CR⁶.

According to the disclosure, Z₄ is N or CR⁶. In some embodiments, Z₄ is N. In other embodiments, Z₄ is CR⁶.

In some embodiments, Z₃ is N and Z₄ is N. In other embodiments, Z₃ is N and Z₄ is CR⁶.

According to the disclosure, each R₇ is independently H, D, C₁₋₆ alkyl, C₃₋₆ cycloalkyl, or haloalkyl. In some embodiments, each R₇ is H. In some embodiments, each R₇ is D. In other embodiments, each R₇ is C₁₋₆ alkyl. In other embodiments, each R₇ is C₃₋₆ cycloalkyl. In other embodiments, each R₇ is haloalkyl.

In some embodiments, at least one R₇ is H. In some embodiments, at least one R₇ is D. In other embodiments, at least one R₇ is C₁₋₆ alkyl. In other embodiments, at least one R₇ is C₃₋₆ cycloalkyl. In other embodiments, at least one R₇ is haloalkyl.

According to the disclosure, q is 1, 2, 3, 4, 5, 6, 7 or 8. In some embodiments, q is 1. In some embodiments, q is 2. In some embodiments, q is 3. In other embodiments, q is 4. In other embodiments, q is 5. In other embodiments, q is 6. In yet other embodiments, q is 7. In yet other embodiments, q is 8.

In some embodiments, the compounds of Formula (I) are represented by compounds of Formula XIIa or formula XIIb:

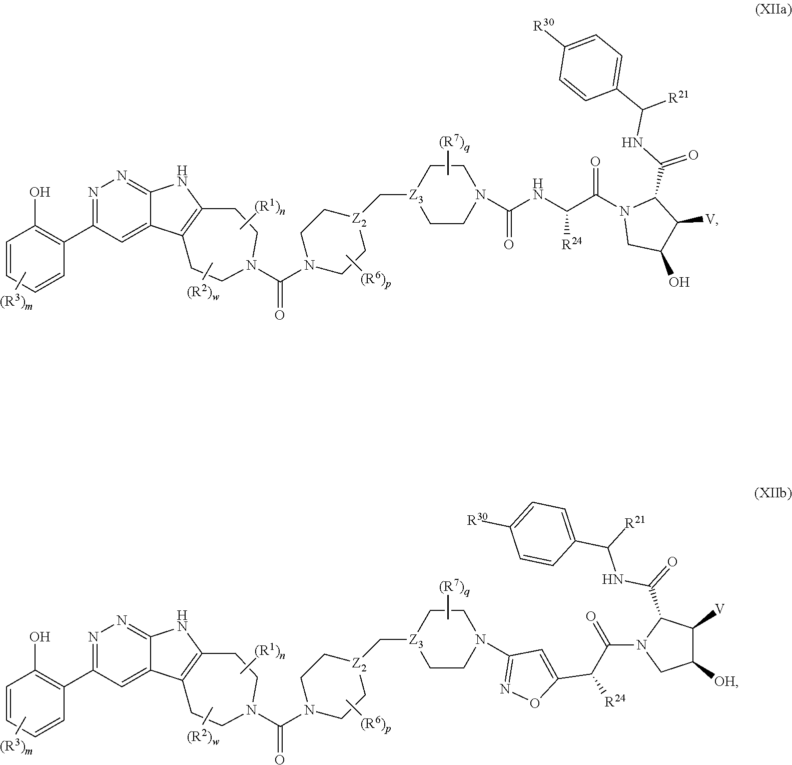

or a pharmaceutically acceptable salt thereof; wherein each (R¹)ₙ, (R²)_{w}, (R³)ₘ, (R⁶)ₚ, (R⁷)_{q}, R²¹, R²⁴, R³⁰, V, Z₁ and Z₂ are defined with respect to Formula I, Formula X and Formula XI above.

In yet further embodiments, the compounds of Formula (I) are:
(S)-3-(6-(4-((4-(3-(2-Hydroxyphenyl)-5,6,7,8,9,10-hexahydropyridazino [4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione;
(S)-3-(6-(4-(((3R,5S)-4-(3-(2-hydroxyphenyl)-5,6,7,8,9,10-hexahydropyridazino [4′,3′:4,5]pyrrolo[2,3-d]azepine-7-carbonyl)-3,5-dimethylpiperazin-1-yl)methyl)piperidin-1-yl)-1-oxoisoindolin-2-yl)piperidine-2,6-dione;
(S)-3-(6-(4-((4-(3-(2-hydroxyphenyl)-5,6,7,8,9,10-hexahydropyridazino[4′,3′:4,5]pyrrolo[2,3-d]azepine-7-carbonyl)piperazin-1-yl)methyl)piperidin-1-yl)-1-oxoisoindolin-2-yl)piperidine-2,6-dione;
(S)-3-(6-(4-((4-(3-(3-Fluoro-2-hydroxyphenyl)-5,6,7,8,9,10-hexahydro-pyridazino [4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione;
(3S)-3-(6-(4-((4-(9-ethyl-3-(3-fluoro-2-hydroxyphenyl)-5,6,7,8,9,10-hexahydro-pyridazino[4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione;
(3S)-3-(6-(4-((4-(3-(3-fluoro-2-hydroxyphenyl)-6,7,8,9,10,11-hexahydro-5H-6,9-epiminocycloocta[4,5]pyrrolo[2,3-c]pyridazine-12-carbonyl)-3,3-dimethylpiperazin-1-yl)methyl)piperidin-1-yl)-1-oxoisoindolin-2-yl)piperidine-2,6-dione;
(3S)-3-(6-(4-((4-(3-(3-fluoro-2-hydroxyphenyl)-5,6,7,8,9,10-hexahydro-6,9-(epiminomethano)cyclohepta[4,5]pyrrolo[2,3-c]pyridazine-12-carbonyl)-3,3-dimethylpiperazin-1-yl)methyl)piperidin-1-yl)-1-oxoisoindolin-2-yl)piperidine-2,6-dione;
or a pharmaceutically acceptable salt thereof.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein). The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

### Pharmaceutical Compositions and Methods of Administration

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (e.g., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or nonaqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, microcrystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (e.g., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%, 50%), 100%, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be affected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

In some embodiments, the compounds or pharmaceutical composition of the present invention are administered by intravenous injection.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; U.S. Pat. Nos. 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc. Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

In certain embodiment, the present invention provides a pharmaceutical composition comprising a compound of bispecific formula, or pharmaceutically acceptable salt thereof.

In certain embodiment, the present invention provides a pharmaceutical composition comprising a compound of bispecific formula for use in degrading a target protein in a cell.

In certain embodiment, a method of degrading a target protein comprising administering to a cell therapeutically effective amount of a bispecific compound, or pharmaceutically acceptable salt, wherein the compound is effective for degrading the target protein.

In certain embodiment, the present invention provides a pharmaceutical composition comprising a compound of bispecific formula, for use in treating or preventing of a disease or disorder in which SMARCA2 and/or SMARCA4 plays a role.

In certain embodiment, the present invention provides a pharmaceutical composition comprising a compound of bispecific formula, for use in treating or preventing of a disease or disorder in which SWI/SNF mutations plays a role.

In certain embodiment, target proteins are SMARCA2, SMARCA4 and/or PB1.

In certain embodiment, target protein complex is SWI/SNF in a cell.

In certain embodiment, diseases or disorders dependent on SMARCA2 or SMARCA4 include cancers.

In certain embodiment, diseases or disorders dependent on SWI/SNF complex include cancers.

Exemplary cancers which may be treated by the present compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

In certain embodiments, the cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

In certain further embodiment, the cancer is a SMARCA2 and/or SMARAC4-dependent cancer.

In certain embodiment, the present invention provides a pharmaceutical composition comprising a compound of bispecific formula for use in the diseases or disorders dependent upon SMARCA2 and/or SMARCA4 is cancer.

Compounds of the disclosure, as well as pharmaceutical compositions comprising them, can be administered to treat any of the described diseases, alone or in combination with a medical therapy. Medical therapies include, for example, surgery and radiotherapy (e.g., gamma-radiation, neutron beam radiotherapy, electron beam radiotherapy, proton therapy, brachytherapy, systemic radioactive isotopes).

In other aspects, compounds of the disclosure, as well as pharmaceutical compositions comprising them, can be administered to treat any of the described diseases, alone or in combination with one or more other agents.

In other methods, the compounds of the disclosure, as well as pharmaceutical compositions comprising them, can be administered in combination with agonists of nuclear receptors agents.

In other methods, the compounds of the disclosure, as well as pharmaceutical compositions comprising them, can be administered in combination with antagonists of nuclear receptors agents.

In other methods, the compounds of the disclosure, as well as pharmaceutical compositions comprising them, can be administered in combination with an anti-proliferative agent.

### Combination Therapies

For treating cancer and other proliferative diseases, the compounds of the invention can be used in combination with chemotherapeutic agents, agonists or antagonists of nuclear receptors, or other anti-proliferative agents. The compounds of the invention can also be used in combination with a medical therapy such as surgery or radiotherapy, e.g., gamma-radiation, neutron beam radiotherapy, electron beam radiotherapy, proton therapy, brachytherapy, and systemic radioactive isotopes. Examples of suitable chemotherapeutic agents include any of: abarelix, aldesleukin, alemtuzumab, alitretinoin, allopurinol, all-trans retinoic acid, altretamine, anastrozole, arsenic trioxide, asparaginase, azacitidine, bendamustine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panobinostat, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinstat and zoledronate.

In some embodiments, the compounds of the invention can be used in combination with a therapeutic agent that targets an epigenetic regulator. Examples of epigenetic regulators include bromodomain inhibitors, the histone lysine methyltransferase inhibitors, histone arginine methyl transferase inhibitors, histone demethylase inhibitors, histone deacetylase inhibitors, histone acetylase inhibitors, and DNA methyltransferase inhibitors. Histone deacetylase inhibitors include, e.g., vorinostat. Histone arginine methyl transferase inhibitors include inhibitors of protein arginine methyltransferases (PRMTs) such as PRMT5, PRMT1 and PRMT4. DNA methyltransferase inhibitors include inhibitors of DNMT1 and DNMT3.

For treating cancer and other proliferative diseases, the compounds of the invention can be used in combination with targeted therapies, including JAK kinase inhibitors (e.g. Ruxolitinib), PI3 kinase inhibitors including PI3K-delta selective and broad spectrum PI3K inhibitors, MEK inhibitors, Cyclin Dependent kinase inhibitors, including CDK4/6 inhibitors and CDK9 inhibitors, BRAF inhibitors, mTOR inhibitors, proteasome inhibitors (e.g. Bortezomib, Carfilzomib), HDAC inhibitors (e.g. panobinostat, vorinostat), DNA methyl transferase inhibitors, dexamethasone, bromo and extra terminal family member (BET) inhibitors, BTK inhibitors (e.g. ibrutinib, acalabrutinib), BCL2 inhibitors (e.g. venetoclax), dual BCL2 family inhibitors (e.g. BCL2/BCLxL), PARP inhibitors, FLT3 inhibitors, or LSD1 inhibitors.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), or PDR001. In some embodiments, the anti-PD-1 monoclonal antibody is nivolumab or pembrolizumab. In some embodiments, the anti-PD1 antibody is pembrolizumab. In some embodiments, the inhibitor of an immune checkpoint molecule is an inhibitor of PD-L1, e.g., an anti-PD-L1 monoclonal antibody. In some embodiments, the anti-PD-L1 monoclonal antibody is atezolizumab, durvalumab, or BMS-935559. In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

The compounds described herein may be prepared according to the following general synthetic procedures.

### Intermediate 1. tert-Butyl 4-(3-(2-hydroxyphenyl)-5,6,7,8,9,10-hexahydropyridazino [4′,3′:4,5]pyrrolo[2,3-d]azepine-7-carbonyl-3,3-dimethylpiperazine-1-carboxylate

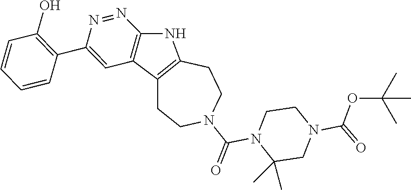 [SMILES: CC(C)(C)OC(=O)N1CCN(C(=O)N2CCc3[nH]c4nnc(-c5ccccc5O)cc4c3CC2)C(C)(C)C1]

### Step 1. tert-Butyl 4-(chlorocarbonyl)-3,3-dimethylpiperazine-1-carboxylate

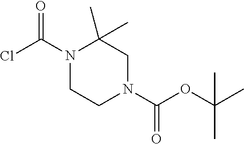 [SMILES: CC(C)(C)OC(=O)N1CCN(C(=O)Cl)C(C)(C)C1]

Triphosgene (415 mg, 1.4 mmol) was added portion wise to a stirring solution of tert-butyl 3,3-dimethylpiperazine-1-carboxylate (500 mg, 2.33 mmol) and pyridine (570 μL, 7.0 mmol) in DCM (20 mL) at 0° C. The reaction was warmed to room temperature and stirred for 2 hours. The product mixture was washed with 1 M HCl aqueous solution (50 mL). The aqueous layer was extracted with DCM (2×50 mL). The combined organic layers were dried over Na₂SO₄, filtered, and concentrated under reduced pressure. The residue obtained was used without further purification. tert-Butyl 4-(chlorocarbonyl)-3,3-dimethylpiperazine-1-carboxylate was obtained as a yellow oil (650 mg, 100%).

### Step 2. tert-Butyl 3-chloro-5,8,9,10-tetrahydropyridazino[4′,3′:4,5]pyrrolo[2,3-d]azepine-7(6H)-carboxylate

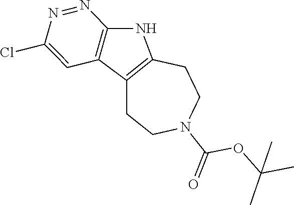 [SMILES: CC(C)(C)OC(=O)N1CCc2[nH]c3nnc(Cl)cc3c2CC1]

Sodium hydride (403 mg, 10.1 mmol, 60% dispersion in mineral oil) was added to a stirring solution of tert-butyl 4-oxoazepane-1-carboxylate (2.05 g, 9.6 mmol) and 4-bromo-6-choropyridazin-3-amine (1.00 g, 4.8 mmol) in THF (36 mL) at 0° C. The reaction mixture was heated to 65° C. and stirred overnight. The product mixture was cooled to 0° C. and quenched with a saturated ammonium chloride aqueous solution (20 mL). The diluted product mixture was transferred to a separatory funnel containing a saturated ammonium chloride aqueous solution (200 mL). The diluted product mixture was extracted with DCM (200 mL×3). The combined organic layers were dried over Na₂SO₄, filtered, and concentrated under reduced pressure. The resulting residue was purified by silica gel flash column chromatography with a gradient of 0-5% MeOH/DCM to obtain tert-butyl 3-chloro-5,8,9,10-tetrahydropyridazino[4′,3′:4,5]pyrrolo[2,3-d]azepine-7(6H)-carboxylate (595 mg, 38%). LCMS calcd for C₁₅H₁₉ClN₄O₂[M+H]⁺: m/z=323.1; Found: 323.0

### Step 3. tert-Butyl 3-(2-hydroxyphenyi)-5,8,9,10-tetrahydropyridazino[4′, 3′:4,5]pyrrolo[2,3-d]azepine-7(6H)-carboxylate

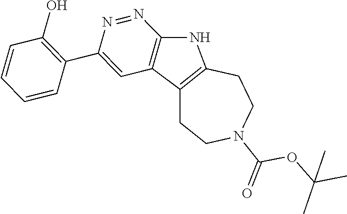 [SMILES: CC(C)(C)OC(=O)N1CCc2[nH]c3nnc(-c4ccccc4O)cc3c2CC1]

A 20 mL scintillation vial was charged with tert-butyl 3-chloro-5,8,9,10-tetrahydropyridazino[4′,3′:4,5]pyrrolo[2,3-d]azepine-7(6H)-carboxylate (554 mg, 1.72 mmol), 2-hydroxyphenylboronic acid (473 mg, 3.43 mmol), XPhos Pd G2 (135 mg, 0.172 mmol), and potassium carbonate (712 mg, 5.15 mmol). The mixture was dissolved in 1,4-dioxane (5.6 mL) and water (1.4 mL). The reaction mixture was sparged with N₂ gas for 5 minutes, sealed, and heated to 80° C. The reaction mixture was stirred for 2 hours at 80° C. The product mixture was diluted with EtOAc (50 mL) and washed with water (100 mL). The aqueous layer was extracted with EtOAc (2×100 mL). The combined organic layers were dried over Na₂SO₄, filtered and concentrated under reduced pressure. The resulting residue was purified by silica gel flash column chromatography with a gradient of 0-80% EtOAc/hexanes to obtain tert-butyl 3-(2-hydroxyphenyl)-5,8,9,10-tetrahydropyridazino[4′,3′:4,5]pyrrolo[2,3-d]azepine-7(6H)-carboxylate (225 mg, 34%), LCMS calcd for C₂₁H₂₄N₄O₃ [M+H]⁺: m/z=381.2; Found: 381.0

### Step 4. 2-(5,6,7,8,9,10-Hexahydropyridazino[4,3′:4,5]pyrrolo[2,3-d]azepin-3-yl)phenol

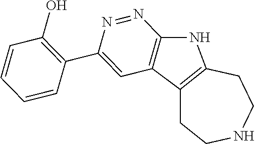 [SMILES: Oc1ccccc1-c1cc2c3c([nH]c2nn1)CCNCC3]

Hydrochloric acid (4.0 M in dioxane, 1.0 mL, 4.0 mmol) was added to a stirring solution of tert-butyl 3-(2-hydroxyphenyl)-5,8,9,10-tetrahydropyridazino[4′,3′:4,5]pyrrolo[2,3-d]azepine-7(6H)-carboxylate (221 mg, 0.581 mmol) in DCM (6 mL) at room temperature. The reaction mixture was stirred overnight. The product mixture was concentrated under reduced pressure to obtain the HCl salt of 2-(5,6,7,8,9,10-hexahydropyridazino[4.3′:4,5]pyrrolo[2,3-d]azepin-3-yl)phenol (184 mg, 100%) as a yellow solid which was used without further purification. LCMS calcd for C₁₆H₁₆N₄O [M+H]⁺: m/z=281.1; Found: 281.0

### Step 5. tert-Butyl 4-(3-(2-hydroxyphenyl-5,6,7,8,9,10-hexahydropyridazino[4′,3′:4,5]pyrrolo[2,3-d]azepine-7-carbonyl)-3,3-dimethylpiperazine-1-carboxylate (Intermediate 1)

tert-Butyl 4-(chlorocarbonyl)-3,3-dimethylpiperazine-1-carboxylate (23.5 mg, 0.085 mmol) in a solution of dimethylacetamide (0.5 mL) was added to a stirring solution of 2-(5,6,7,8,9,10-hexahydropyridazino[4′,3′:4,5]pyrrolo[2,3-]azepin-3-yl)phenol (18 mg, 0,056 mmol), N,N-diisopropylethylamine (39 μL, 0.23 mmol), and 4-dimethylaminopyridine (2.1 mg, 0.017 mmol) in dimethylacetamide (1 mL) at room temperature. The reaction mixture was stirred for 30 minutes. The product mixture was diluted with methanol (3.5 mL) and purified by prep-HPLC (Waters CSH-C18, 5 uM, 30×100 mm, 27-47% MeCN/water (containing 0.1% TFA) over 5 min) to give the TFA salt of tert-butyl 4-(3-(2-hydroxyphenyl)-5,6,7,8,9,10-hexahydro-pyridazino[4′,3′:4,5]pyrrolo[2,3-d]azepine-7-carbonyl)-3,3-dimethylpiperazine-1-carboxylate (13.5 mg, 38%). LCMS calcd for C₂₈H₃₆N₆O₄[M+H]⁺: m/z=521.3; Found: 521.1

### Intermediates 2-3

The intermediates shown below in Table 1 were prepared by methods analogous to that described for the preparing Intermediate 1 using the appropriate starting materials.

TABLE 1
Intermediates 2-3

| | | | Calcd. | Found |
|---|---|---|---|---|
| | | | (M + H)⁺ | (M + H)⁺ |
| Int. | Structure | Name | m/z | m/z |
| 2 | 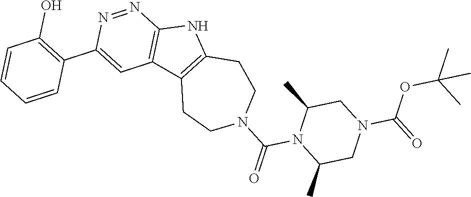 [SMILES: C[C@@H]1CN(C(=O)OC(C)(C)C)C[C@H](C)N1C(=O)N1CCc2[nH]c3nnc(-c4ccccc4O)cc3c2CC1] | tert-butyl (3R,5S)-4-(3-(2- hydroxyphenyl)- 5,6,7,8,9,10-hexahydro- pyridazino [4′,3′:4,5]pyrrolo[2,3- d]azepine-7-carbonyl)-3,5- dimethyl-piperazine-1- carboxylate | 521.3 | 521.1 |
| 3 | 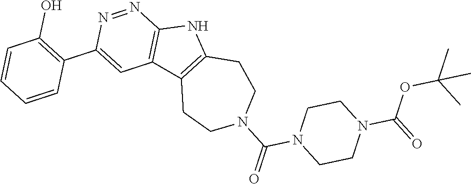 [SMILES: CC(C)(C)OC(=O)N1CCN(C(=O)N2CCc3[nH]c4nnc(-c5ccccc5O)cc4c3CC2)CC1] | tert-butyl 4-(3-(2- hydroxyphenyl)- 5,6,7,8,9,10-hexahydro- pyridazino [4′,3′:4,5]pyrrolo[2,3- d]azepine-7-carbonyl) piperazine-1-carboxylate | 493.3 | 493.1 |

### Example 1. (S)-3-(6-(4-((4-(3-(2-Hydroxyphenyl)-5,6,7,8,9,10-hexahydropyridazino [4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione

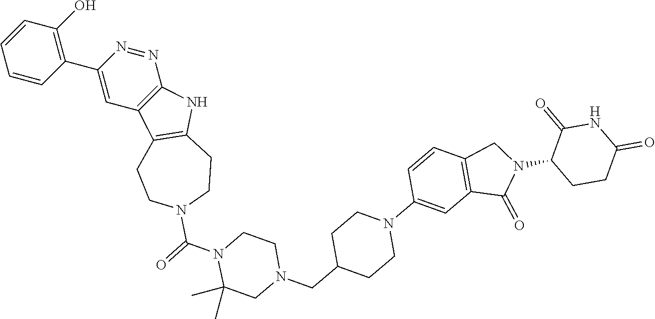 [SMILES: CC1(C)CN(CC2CCN(c3ccc4c(c3)C(=O)N([C@H]3CCC(=O)NC3=O)C4)CC2)CCN1C(=O)N1CCc2[nH]c3nnc(-c4ccccc4O)cc3c2CC1]

### Step 1. (2,2-Dimethylpiperazin-1-yl)(3-(2-hydroxyphenyl)-5,8,9,10-tetrahydropyridazino [4′,3′:4,5]pyrrolo[2,3-d]azepin-7(6H)-yl)methanone

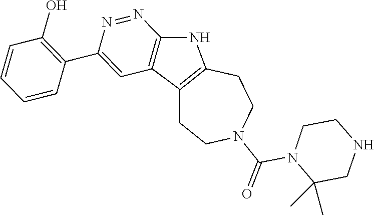 [SMILES: CC1(C)CNCCN1C(=O)N1CCc2[nH]c3nnc(-c4ccccc4O)cc3c2CC1]

Trifluoroacetic acid (600 μL, 7.8 mmol) was added to a stirring solution of tert-butyl 4-(3-(2-hydroxyphenyl)-5,6,7,8,9,10-hexahydropyridazino[4′,3′:4,5]pyrrolo[2,3-d]azepine-7-carbonyl)-3,3-dimethylpiperazine-1-carboxylate (13.5 mg, 0.022 mmol) in DCM (2 mL) at room temperature. The reaction mixture was stirred 30 minutes then concentrated under reduced pressure to obtain the TFA salt of (2,2-dimethylpiperazin-1-yl)(3-(2-hydroxyphenyl)-5,8,9,10-tetrahydropyridazino[4′,3′:4,5]pyrrolo[2,3-d]azepin-7(6H)-yl)methanone (11.3 mg, 100%) as a yellow residue which was used without further purification. LCMS calcd for C₂₃H₂₈N₆O₂ [M+H]⁺: m/z=421.2; Found: 421.1

### Step 2. (S)-3-(6-(4-((4-(3-(2-Hydroxyphenyl)-5,6,7,8,9,10-hexahydropyridazino [4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione

N,N-Diisopropylethylamine (10.3 μL, 0.057 mmol) was added to a stirring solution of (2,2-dimethylpiperazin-1-yl)(3-(2-hydroxyphenyl)-5,8,9,10-tetrahydropyridazino[4′,3′:4,5]pyrrolo[2,3-d]azepin-7(6H)-yl)methanone (8.0 mg, 0.019 mmol) and (S)-1-(2-(2,6-dioxopiperidin-3-yl)-3-oxoisoindolin-5-yl)piperidine-4-carbaldehyde (10.1 mg, 0.029 mmol) in DMF (1 mL). The reaction mixture was stirred for 30 minutes. Sodium triacetoxyborohydride (7.4 mg, 0.057 mmol) was added to the reaction mixture. The reaction mixture was heated to 35° C. and stirred for 1 hour. The product mixture was diluted with water (1 mL) and acetonitrile (3 mL) and purified by prep-HPLC (Waters CSH-C18, 5 uM, 30×100 mm, 7.8-27.8% MeCN/water (containing 0.1% TFA) over 5 min) to give the TFA salt of (S)-3-(6-(4-((4-(3-(2-hydroxyphenyl)-5,6,7,8,9,10-hexahydropyridazino[4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione (2.3 mg, 12%). LCMS calcd for C₄₂H₄₉N₉O₅ [M+H]⁺: m/z=760.4; Found: 760.3

### Examples 2-3

Examples shown below in Table 2 were prepared as TFA salts by the method used in preparing Example 1 using the appropriate intermediates and starting materials.

TABLE 2
Examples 2-3

| | | Calcd. | Found |
|---|---|---|---|
| Ex. | Structure | (M + H)⁺ m/z | (M + H)⁺ m/z |
| 2 | 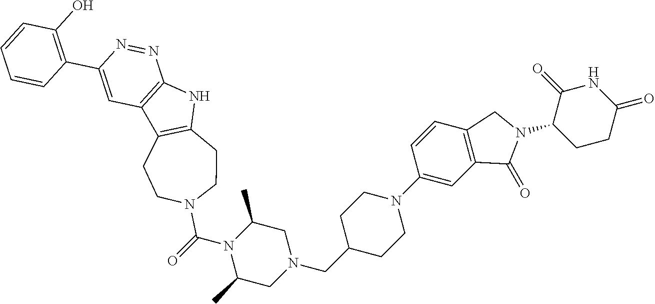 [SMILES: C[C@@H]1CN(CC2CCN(c3ccc4c(c3)C(=O)N([C@H]3CCC(=O)NC3=O)C4)CC2)C[C@H](C)N1C(=O)N1CCc2[nH]c3nnc(-c4ccccc4O)cc3c2CC1] | 760.4 | 760.3 |
| | (S)-3-(6-(4-(((3R,5S)-4-(3-(2-hydroxyphenyl)-5,6,7,8,9,10- | | |
| | hexahydropyridazino[4′,3′:4,5]pyrrolo[2,3-d]azepine-7- | | |
| | carbonyl)-3,5-dimethylpiperazin-1-yl)methyl)piperidin-1-yl)- | | |
| | 1-oxoisoindolin-2-yl)piperidine-2,6-dione | | |
| 3 | 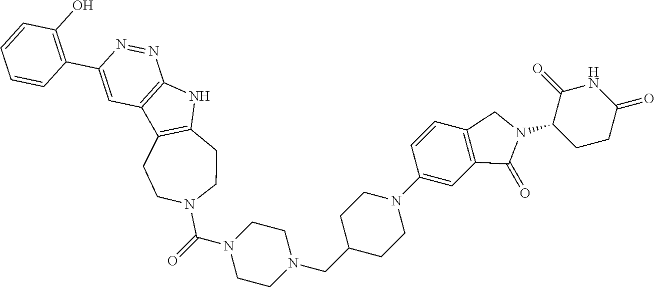 [SMILES: O=C1CC[C@H](N2Cc3ccc(N4CCC(CN5CCN(C(=O)N6CCc7[nH]c8nnc(-c9ccccc9O)cc8c7CC6)CC5)CC4)cc3C2=O)C(=O)N1] | 732.4 | 732.3 |
| | (S)-3-(6-(4-((4-(3-(2-hydroxyphenyl)-5,6,7,8,9,10- | | |
| | hexahydropyridazino[4′,3′:4,5]pyrrolo[2,3-d]azepine-7- | | |
| | carbonyl)piperazin-1-yl)methyl)piperidin-1-yl)-1- | | |
| | oxoisoindolin-2-yl)piperidine-2,6-dione | | |

### Intermediate 4. tert-Butyl 3-ethyl-4-oxoazepane-1-carboxylate

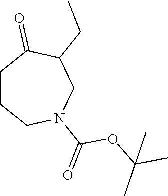 [SMILES: CCC1CN(C(=O)OC(C)(C)C)CCCC1=O]

Lithium bis(trimethylsilyl)amide (1.0 M, 3.16 mL, 3.16 mmol) was added to a stirring solution of tert-butyl 4-oxoazepane-1-carboxylate (450 mg, 2.11 mmol) in THF (10.5 mL) at −78° C. The reaction mixture was stirred for 1 hour at −78° C. Iodoethane (509 μL, 6.33 mmol) was added to the reaction mixture at −78° C. The reaction mixture was removed from the cooling batch and allowed to warm to room temperature. The reaction mixture was stirred at room temperature for 16 hours. The product mixture was transferred to a separatory funnel containing a saturated ammonium chloride aqueous solution (100 mL). The diluted product mixture was extracted with DCM (100 mL×3). The combined organic layers were dried over Na₂SO₄, filtered, and concentrated under reduced pressure. The resulting residue was purified by silica gel flash column chromatography with a gradient of 0-40% EtOAc/Hexanes to obtain tert-butyl 3-ethyl-4-oxoazepane-1-carboxylate (294 mg, 58%). LCMS calcd for C₁₃H₂₃NO₃ [M+H−C₄H₉]⁺: m/z=186.1; Found: 186.1. LCMS calcd for C₁₃H₂₃NO₃ [M+H−C₅H₉O₂]⁺: m/z=142.1; Found: 142.0

### Intermediate 5. tert-Butyl 3-oxo-9-azabicyclo[4.2.1]nonane-9-carboxylate

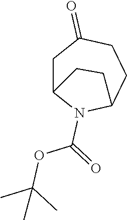 [SMILES: CC(C)(C)OC(=O)N1C2CCC(=O)CC1CC2]

### Step 1. 9-tert-Butyl) 3-ethyl 4-oxo-azabicyclo[4.2.1]nonane-3,9-dicarboxylate

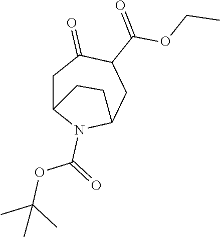 [SMILES: CCOC(=O)C1CC2CCC(CC1=O)N2C(=O)OC(C)(C)C]

Boron trifluoride diethyl etherate (0.22 mL, 1.78 mmol) was added to a stirring solution of tert-butyl-3-oxo-8-azabicyclo[3.2.1]octane-8-carboxylate (400 mg, 1.78 mmol) in DCM (12 mL) at −78° C. The reaction mixture was stirred for 5 minutes. Ethyl diazoacetate (2.15 mL, 1.78 mmol, 13% by weight in DCM) was added to the reaction mixture at −78° C. The reaction mixture was warmed to 0° C. and stirred for 30 minutes. The product mixture was quenched with a saturated sodium bicarbonate aqueous solution (10 mL) and stirred for 10 minutes. The quenched product mixture was transferred to a separatory funnel containing a saturated sodium bicarbonate aqueous solution (100 mL) and extracted with DCM (100 mL×3). The combined organic layers were dried over Na₂SO₄, filtered, and concentrated under reduced pressure. The resulting residue was purified by silica gel flash column chromatography with a gradient of 0-40% EtOAc/hexanes to obtain 9-(tert-butyl) 3-ethyl 4-oxo-9-azabicyclo[4.2.1]nonane-3,9-dicarboxylate (457 mg, 83%). LCMS calcd for C₁₆H₂₅NO₅ [M+H−C₅H₉O₂]⁺: m/z=212.1; Found: 212.1

### Step 2. Synthesis of tert-butyl 3-oxo-9-azabicyclo[4.2.1]nonane-9-carboxylate

Sodium hydroxide (212 mg, 5.3 mmol) was added to a stirring solution of obtain 9-(tert-butyl) 3-ethyl 4-oxo-9-azabicyclo[4.2.1]nonane-3,9-dicarboxylate (574 mg, 1.84 mmol) in 1,4-dioxane (5 mL) and water (2.5 mL) at room temperature. The reaction mixture was stirred at room temperature overnight. The product mixture was acidified with 1 M HCl aqueous solution to a pH of 5. The acidified product mixture was extracted with DCM. The combined organic layers were dried over Na₂SO₄, filtered, and concentrated under reduced pressure to obtain tert-butyl 3-oxo-9-azabicyclo[4.2.1]nonane-9-carboxylate. The resulting residue was used directly in the next step. LCMS calcd for C₁₃H₂₁NO₃ [M+H−C₄H₉]⁺: m/z=184.1; Found: 184.0. LCMS calcd for C₁₃H₂₁NO₃ [M+H−C₅H₉O₂]⁺: m/z=140.1; Found: 140.0

### Intermediate 6. (2,2-Dimethylpiperazin-1-yl)(3-(3-fluoro-2-hydroxyphenyl)-5,8,9,10-tetrahydropyridazino[4′,3′:4,5]pyrrolo[2,3-d]azepin-7(6H)-yl)methanone

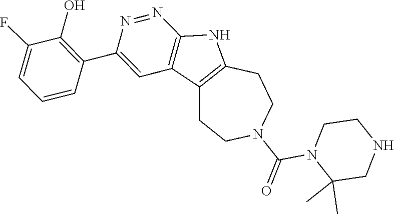 [SMILES: CC1(C)CNCCN1C(=O)N1CCc2[nH]c3nnc(-c4cccc(F)c4O)cc3c2CC1]

### Step 1. tert-Butyl 3-(3-fluoro-2-hydroxyphenyl)5,8,9,10-tetrahydropyridazino[4′,3′:4,5]pyrrolo[2,3-d]azepine-7(61)-carboxylate

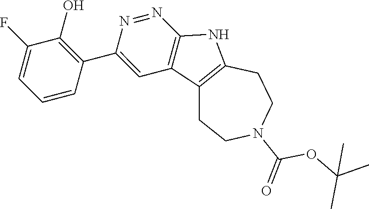 [SMILES: CC(C)(C)OC(=O)N1CCc2[nH]c3nnc(-c4cccc(F)c4O)cc3c2CC1]

A 20 mL scintillation vial was charged with tert-butyl 3-chloro-5,8,9,10-tetrahydropyridazino[4′,3′:4,5]pyrrolo[2,3-d]azepine-7(6H)-carboxylate (595 mg, 1.84 mmol), 3-fluoro-2-hydroxyphenylboronic acid (575 mg, 3.69 mmol), [1,1′-bis(diphenylphosphino)-ferrocene]dichloropalladium (II) complex with DCM (226 mg, 0.27 mmol), and potassium carbonate (1.02 g, 7.37 mmol). The mixture was dissolved in 1,4-dioxane (7.3 mL) and water (1.9 mL). The reaction mixture was sparged with N₂ gas for 5 minutes, sealed, and heated to 100° C. The reaction mixture was stirred for 2 hours at 100° C. The product mixture was transferred to a separatory funnel containing saturated sodium bicarbonate aqueous solution (100 mL). The aqueous layer was extracted with DCM (3×100 mL). The combined organic layers were dried over Na₂SO₄, filtered and concentrated under reduced pressure. The resulting residue was purified by silica gel flash column chromatography with a gradient of 0-100% EtOAc/hexanes to obtain tert-butyl 3-(3-fluoro-2-hydroxyphenyl)-5,8,9,10-tetrahydropyridazino[4′,3′:4,5]pyrrolo [2,3-d]azepine-7(6H)-carboxylate (356 mg, 48%). LCMS calcd for C₂₁H₂₃FN₄O₃ [M+H]⁺: m/z=399.2; Found: 399.1

### Step 2. tert-Butyl 4-(3-(3 fluoro-2-hydroxyphenyl)-5,6,7,8,9,70-hexahydropyridazino [4′, 3′:4,5]pyrrolo[2,3-d]azepine-7-carbonyl)-3,3-dimethylpiperazine-1-carboxylate

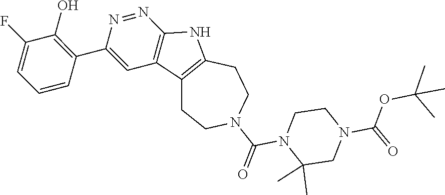 [SMILES: CC(C)(C)OC(=O)N1CCN(C(=O)N2CCc3[nH]c4nnc(-c5cccc(F)c5O)cc4c3CC2)C(C)(C)C1]

tert-Butyl 4-(chlorocarbonyl)-3,3-dimethylpiperazine-1-carboxylate (78.6 mg, 0.284 mmol) in a solution of dimethylacetamide (0.5 mL) was added to a stirring solution of tert-butyl 3-(3-fluoro-2-hydroxyphenyl)-5,8,9,10-tetrahydropyridazino[4′,3′:4,5]pyrrolo[2,3-d]azepine-7(6H)-carboxylate (63.4 mg, 0.189 (o), N,N-diisopropylethylamine (132 μL, 0.76 mmol), and 4-dimethylaminopyridine (6.9 mg, 0,057 mmol) in dimethylacetamide (2 mL) at room temperature. The reaction mixture was stirred for 30 minutes. The product mixture was diluted with methanol (3.5 mL) and purified by prep-HPLC (Waters CSH-C18, 5 uM, 30×100 mm, 27.8-47.8% MeCN/water (containing 0.1% TFA) over 5 min) to give the TFA salt of tert-butyl 4-(3-(3-fluoro-2-hydroxyphenyl)-5,6,7,8,9,10-hexahydropyridazino[4′,3′:4,5]pyrrolo[2,3-d]azepine-7-carbonyl)-3,3-dimethylpiperazine-1-carboxylate (35 mg, 29%). ¹H NMR (400 MHz, MeOD) δ 8.48 (s, 1H), 7.48 (d, J=8.0 Hz, 1H), 7.39-7.30 (m, 1H), 7.07 (t, J=6.6 Hz, 1H), 3.79-3.70 (m, 4H), 3.55 (s, 2H), 3.40-3.36 (m, 2H), 3.28-3.11 (m, 6H), 1.48 (s, 9H), 1.30 (s, 6H). LCMS calcd for C₂₈H₃₅FN₆O₄[M+H]⁺: m/z=539.3; Found: 539.2.

### Step 3. (2,2-Dimethylpiperazin-1-yl)(3-(3-fluoro-2-hydroxyphenyl)-5,8,9,10-tetrahydropyridazino[4′,3′:4,5]pyrrolo[2,3-d]azepin-7(6H)-yl)methanone

Trifluoroacetic acid (600 μL, 7.8 mmol) was added to a stirring solution of tert-butyl 4-(3-(3-fluoro-2-hydroxyphenyl)-5,6,7,8,9,10-hexahydropyridazino[4′,3′:4,5]pyrrolo[2,3-d]azepine-7-carbonyl)-3,3-dimethylpiperazine-1-carboxylate (35 mg, 0.022 mmol) in DCM (2 mL) at room temperature. The reaction mixture was stirred 30 minutes then concentrated under reduced pressure to obtain the TFA salt of (2,2-dimethylpiperazin-1-yl)(3-(3-fluoro-2-hydroxyphenyl)-5,8,9,10-tetrahydropyridazino[4′,3′:4,5]pyrrolo[2,3-d]azepin-7(6H)-yl)methanone (29.5 mg, 100%) as a yellow residue which was used without further purification. LCMS calcd for C₂₃H₂₇FN₆O₂ [M+H]⁺: m/z=439.2; Found: 439.2

### Intermediates 7-9

The intermediates shown below in Table 3 were prepared by methods analogous to that described for the preparing Intermediate 6 using the appropriate starting materials.

TABLE 3
Intermediates 7-9

| | | | Calcd. | Found |
|---|---|---|---|---|
| Int. | Structure | Name | (M + H)⁺ m/z | (M + H)⁺ m/z |
| 7 | 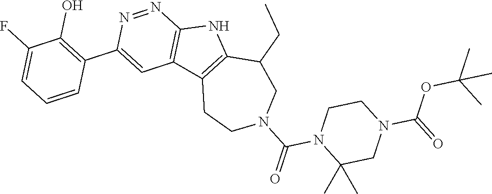 [SMILES: CCC1CN(C(=O)N2CCN(C(=O)OC(C)(C)C)CC2(C)C)CCc2c1[nH]c1nnc(-c3cccc(F)c3O)cc21] | tert-butyl 4-(9-ethyl-3- (3-fluoro-2- hydroxyphenyl)- 5,6,7,8,9,10- hexahydropyridazino [4′,3′:4,5]pyrrolo[2,3- d]azepine-7-carbonyl)- 3,3-dimethyl- piperazine-1- carboxylate | 567.3 | 567.2 |
| 8 | 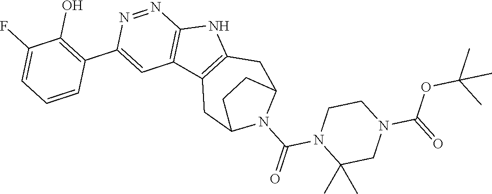 [SMILES: CC(C)(C)OC(=O)N1CCN(C(=O)N2C3CCC2Cc2c([nH]c4nnc(-c5cccc(F)c5O)cc24)C3)C(C)(C)C1] | tert-butyl 4-(3-(3- fluoro-2- hydroxyphenyl)- 6,7,8,9,10,11- hexahydro-5H-6,9- epiminocycloocta[4,5] pyrrolo[2,3- c]pyridazine-12- carbonyl)-3,3- dimethylpiperazine-1- carboxylate | 565.3 | 565.2 |
| | ¹H NMR (400 MHz, MeOD) δ 8.47 (s, 1H), | | | |
| | 7.43 (dd, J = 7.8, 1.4 Hz, 1H), 7.41 (dt, J = | | | |
| | 9.4, 1.5 Hz, 1H), 7.08 (td, J = 8.0, 4.8 Hz, | | | |
| | 1H), 4.61-4.53 (m, 2H), 3.82 (dd, J = 17.5, | | | |
| | 3.8 Hz, 1H), 3.70-3.61 (m, 1H), 3.57- | | | |
| | 3.44 (m, 1H), 3.44-3.31 (m, 2H), 3.27- | | | |
| | 3.06 (m, 4H), 2.34-2.23 (m, 2H), 1.74- | | | |
| | 1.61 (m, 2H), 1.48 (s, 9H), 1.32 (s, 3H), 1.28 | | | |
| | (s, 3H). | | | |
| 9 | 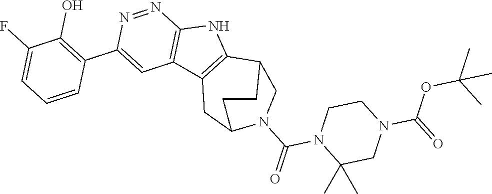 [SMILES: CC(C)(C)OC(=O)N1CCN(C(=O)N2CC3CCC2Cc2c3[nH]c3nnc(-c4cccc(F)c4O)cc23)C(C)(C)C1] | tert-butyl 4-(3-(3- fluoro-2- hydroxyphenyl)- 5,6,7,8,9,10- hexahydro-6,9- (epiminomethano) cyclohepta[4,5]pyrrolo[2,3- c]pyridazine-12- carbonyl)-3,3- dimethylpiperazine-1- carboxylate | 565.3 | 565.2 |

### Example 4. (S)-3-(6-(4-((4-(3-(3-Fluoro-2-hydroxyphenyl)-5,6,7,8,9,10-hexahydro-pyridazino[4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione

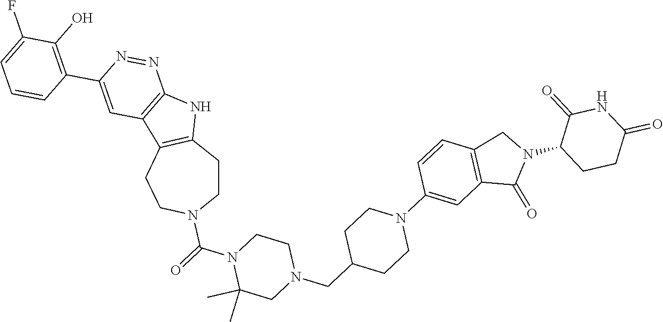 [SMILES: CC1(C)CN(CC2CCN(c3ccc4c(c3)C(=O)N([C@H]3CCC(=O)NC3=O)C4)CC2)CCN1C(=O)N1CCc2[nH]c3nnc(-c4cccc(F)c4O)cc3c2CC1]

N,N-Diisopropylethylamine (25.7 μL, 0.143 mmol) was added to a stirring solution of (2,2-dimethylpiperazin-1-yl)(3-(3-fluoro-2-hydroxyphenyl)-5,8,9,10-tetrahydropyridazino [4′,3′:4,5]pyrrolo[2,3-d]azepin-7(6H)-yl)methanone (20.9 mg, 0.048 mmol) and (S)-1-(2-(2,6-dioxopiperidin-3-yl)-3-oxoisoindolin-5-yl)piperidine-4-carbaldehyde (20.3 mg, 0.057 mmol) in DMF (1 mL). The reaction mixture was stirred for 30 minutes. Sodium triacetoxyborohydride (18.4 mg, 0.143 mmol) was added to the reaction mixture. The reaction mixture was heated to 35° C. and stirred for 1 hour. The product mixture was diluted with water (1 mL) and acetonitrile (3 mL) and purified by prep-HPLC (Waters CSH-C18, 5 uM, 30×100 mm, 7.8-27.8% MeCN/water (containing 0.1% TFA) over 5 min) to give the TFA salt of (S)-3-(6-(4-((4-(3-(3-fluoro-2-hydroxyphenyl)-5,6,7,8,9,10-hexahydropyridazino[4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione (6.2 mg, 13%). LCMS calcd for C₄₂H₄₈FN₉O₅ [M+H]⁺: m/z=778.4; Found: 778.3.

### Examples 5-7

Examples shown below in Table 4 were prepared as TFA salts by the method used in preparing Example 4 using the appropriate intermediates and starting materials.

TABLE 4
Examples 5-7

| | | Calcd. | Found |
|---|---|---|---|
| Ex. | Structure | (M + H)⁺ m/z | (M + H)⁺ m/z |
| 5 | 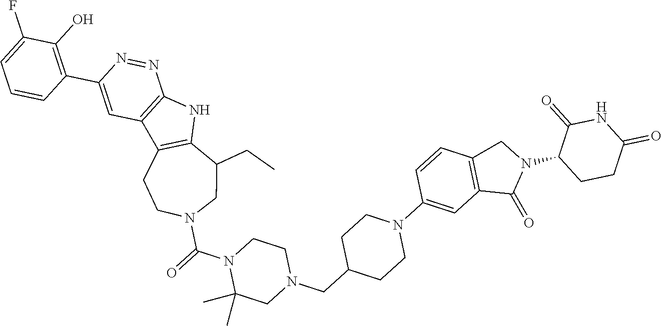 [SMILES: CCC1CN(C(=O)N2CCN(CC3CCN(c4ccc5c(c4)C(=O)N([C@H]4CCC(=O)NC4=O)C5)CC3)CC2(C)C)CCc2c1[nH]c1nnc(-c3cccc(F)c3O)cc21] | 806.4 | 806.3 |
| | (3S)-3-(6-(4-((4-(9-ethyl-3-(3-fluoro-2-hydroxyphenyl)- | | |
| | 5,6,7,8,9,10-hexahydropyridazino[4′,3′:4,5]pyrrolo[2,3- | | |
| | d]azepine-7-carbonyl)-3,3-dimethylpiperazin-1- | | |
| | yl)methyl)piperidin-1-yl)-1-oxoisoindolin-2-yl)piperidine-2,6- | | |
| | dione | | |
| 6 | 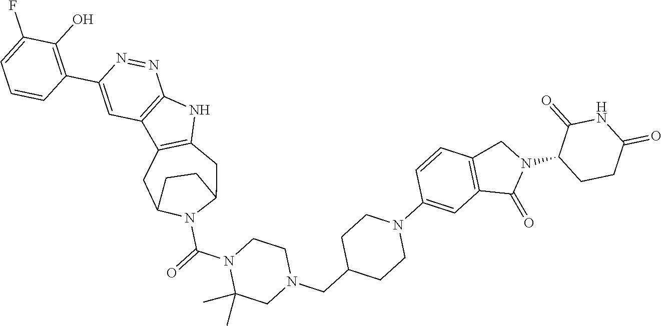 [SMILES: CC1(C)CN(CC2CCN(c3ccc4c(c3)C(=O)N([C@H]3CCC(=O)NC3=O)C4)CC2)CCN1C(=O)N1C2CCC1Cc1c([nH]c3nnc(-c4cccc(F)c4O)cc13)C2] | 804.4 | 804.4 |
| | (3S)-3-(6-(4-((4-(3-(3-fluoro-2-hydroxyphenyl)-6,7,8,9,10,11- | | |
| | hexahydro-5H-6,9-epiminocycloocta[4,5]pyrrolo[2,3- | | |
| | c]pyridazine-12-carbonyl)-3,3-dimethylpiperazin-1- | | |
| | yl)methyl)piperidin-1-yl)-1-oxoisoindolin-2-yl)piperidine-2,6- | | |
| | dione | | |
| 7 | 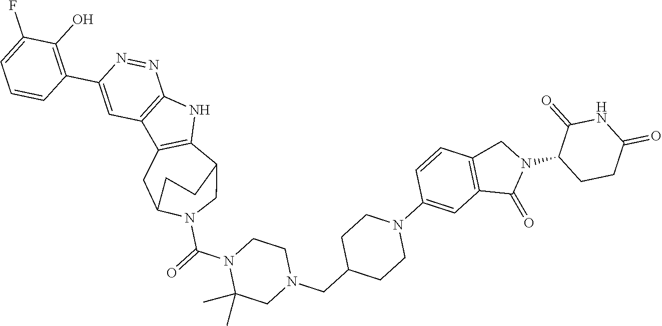 [SMILES: CC1(C)CN(CC2CCN(c3ccc4c(c3)C(=O)N([C@H]3CCC(=O)NC3=O)C4)CC2)CCN1C(=O)N1CC2CCC1Cc1c2[nH]c2nnc(-c3cccc(F)c3O)cc12] | 804.3 | 804.3 |
| | (3S)-3-(6-(4-((4-(3-(3-fluoro-2-hydroxyphenyl)-5,6,7,8,9,10- | | |
| | hexahydro-6,9-(epiminomethano)cyclohepta[4,5]pyrrolo[2,3- | | |
| | c]pyridazine-12-carbonyl)-3,3-dimethylpiperazin-1- | | |
| | yl)methyl)piperidin-1-yl)-1-oxoisoindolin-2-yl)piperidine-2,6- | | |
| | dione | | |

### Example A. SMARCA2 HiBiT and SMARCA4 HiBiT Degradation Assay

### Preparation of SMARCA2/4-HiBiT Knock-In Cells

HiBiT peptide knock-in of SMARCA2 in LgBiT expressing HEK293T cells was performed by CRISPR-mediated tagging system as described Promega. The homozygous HiBiT knock-in on c-terminus SMARCA2 was confirmed by sanger sequence. SMARCA2-HiBiT knock-in Hela monoclonal cell (CS302366) and SMARCA4-HiBiT knock-in Hela monoclonal cell (CS3023226) were purchased from Promega. The heterozygous HiBiT-knock-in was confirmed by sanger sequence in both SMARCA2-HiBiT and SMARCA4-HiBiT monoclonal cells.

### SMARCA2 HiBiT and SMARCA4 HiBiT Degradation Assay in HeLa Cells

Compounds were dissolved in DMSO to make 10 mM stock and 3-fold series dilutions were further conducted keeping the highest concentration 10 μM. NCIH1693 and NCIH520 cells were maintained in PRMI 1640 medium (Corning Cellgro, Catalog #:10-040-CV) supplemented with 10% v/v FBS (GE Healthcare, Catalog #: SH30910.03) by splitting 1:3 twice a week.

Dispense 10 ul aliquot of prepared Hela-SMARCA2-HiBiT or Hela-SMARCA4-HiBiT cells (1:1 ratio of cells:Trypan Blue (#1450013, Bio-Rad)) onto cell counting slide (#145-0011, Bio-Rad) and obtain cell density and cell viability using cell counter (TC20, Bio-Rad). Remove appropriate volume of resuspended cells from culture flask to accommodate 2500 cells/well @20 mL/well. Transfer Hela-HiBiT cells to 50 mL conical (#430290, Corning). Spin down at 1000 rpm for 5 min using tabletop centrifuge (SPINCHRON 15, Beckman). Discard supernatant and resuspend cell pellet in modified EMEM (#30-2003, ATCC) cell culture media containing 10% FBS (F2422-500 ML, Sigma), and 1× Penicillin/Streptomycin (200 g/L) (30-002-CI, Corning) to a cell density of 125,000 cells/mL. Dispense 20 mL of resuspended Hela-HiBit cells per well in 384-well TC treated plate (#12-565-343, Thermo Scientific) using standard cassette (#50950372, Thermo Scientific) on Multidrop Combi (#5840310, Thermo Scientific) inside laminar flow cabinet. Dispense test compounds onto plates using digital liquid dispenser (D300E, Tecan). Incubate plates in humidified tissue culture incubator @37° C. for 18 hours. Add 20 mL of prepared Nano-Glo® HiBiT Lytic detection buffer (N3050, Promega) to each well of 384-well plate using small tube cassette (#24073295, Thermo Scientific) on Multidrop Combi, incubate @RT for 30-60 min. Read plates on microplate reader (Envision 2105, PerkinElmer) using 384 well Ultra-Sensitive luminescence mode. Raw data files and compound information reports are swept into centralized data lake and deconvoluted using automated scripts designed by TetraScience, Inc. Data analysis, curve-fitting and reporting done in Dotmatics Informatics Suite using Screening Ultra module.

Results are summarized below in Table 5. In Table 5, A=DC₅₀<0.1 μM; B=0.1 μM≤DC₅₀<1 μM and C=DC₅₀>1 μM. In Table 5, A=Dₘₐₓ>75% and B=50%<Dₘₐₓ≤75% and C=Dₘₐₓ<50%.

TABLE 5
Biological Data

| | SMARCA2 | SMARCA2 | SMARCA4 | SMARCA4 |
|---|---|---|---|---|
| Example | DC₅₀ (nM) | Dₘₐₓ % | DC₅₀ (nM) | Dₘₐₓ % |
| 1 | A | A | A | A |
| 2 | A | A | A | A |
| 3 | A | A | A | A |
| 4 | A | B | C | C |
| 5 | B | B | C | C |
| 6 | A | A | B | B |
| 7 | B | C | B | C |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula (I): 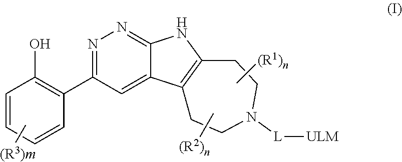
or a pharmaceutically acceptable salt thereof; wherein
each R¹ and R² is independently H, D, OR^{a}, C₁-C₈ alkoxy, C₁-C₈ alkyl, haloalkyl, —C₃-C₈ cycloalkyl, —C₃-C₁₀ cycloalkenyl, aryl, heteroaryl, heterocycloalkyl, heterocycloalkenyl, or (C₁-C₆-alkyl)-R^{e}; wherein said C₁-C₈ alkoxy, C₁-C₈ alkyl, haloalkyl, C₃-C₈ cycloalkyl, —C₃-C₁₀ cycloalkenyl, aryl, heteroaryl, heterocycloalkyl, heterocycloalkenyl, or (C₁-C₆-alkyl)-R^{e} are optionally substituted by 1-6 R^{f} groups; or
an R¹ and an R² may optionally be connected to form a 4-8 membered cycloalkyl or heterocycloalkyl ring;
R^{e} is C₃-C₈ cycloalkyl, heterocycloalkyl wherein the heterocycloalkyl is attached to (C₁-C₆-alkyl) through a carbon atom or a sulfur atom of the heterocycloalkyl group, cycloalkenyl, heterocycloalkenyl wherein the heterocycloalkenyl is attached to (C₁-C₆-alkyl) through a carbon atom or a sulfur atom of the heterocycloalkenyl group, aryl, or heteroaryl, and each C₃-C₈ cycloalkyl, heterocycloalkyl, cycloalkenyl, heterocycloalkenyl, aryl, or heteroaryl is optionally substituted by 1-6 R^{f} groups;
each R^{f} is independently H, D, oxo, halogen, C₁-C₈ alkoxy, C₁-C₈ alkyl, haloalkyl, —OH, —CN, —NO2, —C2-C6 alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF5, —P(O)R^{b}R^{b}, —P(O)R^{c}R^{d}, —P(O)(OR^{b})(OR^{b}), —B(OR)(OR^{d}), —S(O)2R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d};
each R^{a} is independently H, D, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)R^{c}R^{d}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocycloalkenyl group;
each R³ is independently H, D, halo, C₁₋₆ alkyl, haloalkyl, or C₃₋₆ cycloalkyl;
n is 1, 2, 3 or 4;
m is 1, 2, 3 or 4;
w is 1, 2, 3, or 4;
L is a linking group to ULM; and
ULM is a CRBN binding moiety or a VHL binding moiety.

2. The compound according to claim 1, wherein n is 1 or 2.

3-9. (canceled)

10. The compound according to claim 1, wherein m is 1 and R³ is halo.

11. (canceled)

12. The compound according to claim 1, wherein each R¹ and R² is C₁₋₄ alkyl.

13. The compound according to claim 1, wherein L is represented by the formula:
-(A)_{q}-,
wherein:
q is an integer from 1 to 14;
each A is independently selected from the group consisting of CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a}, (CR^{1a}R^{1b})₁₋₄, —(CR^{1a}R^{1b})₁₋₄O(CR^{1a}R^{1b})₁₋₄, —(CR^{1a}R^{1b})₁₋₄S(CR^{1a}R^{1b})₁₋₄, —(CR^{1a}R^{1b})₁₋₄NR^{1c}(CR^{1a}R^{1b})₁₋₄, NR^{1c}C(═NCN)NR^{1d}, NR^{1c}C(═NCN), NR^{1c}C(═CNO₂)NR^{1d}, 3-11 membered cycloalkyl, optionally substituted with 1-6 R^{1a} or R^{1b} groups, 3-11 membered heterocyclyl optionally substituted with 1-6 R^{1a} or R^{1b} groups, aryl optionally substituted with 1-6 R^{1a} or R^{1b} groups, or heteroaryl optionally substituted with 1-6 R^{1a} or R^{1b} groups,
wherein R^{1a}, R^{1b}, R^{1c}, R^{1d} and R^{1e} are each independently, —H, D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 1-4 R^{1e} groups.

14. The compound according to claim 13 wherein q is an integer from 1 to 4.

15. The compound according to claim 1, wherein L is 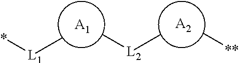
wherein
* is the point of attachment to N; and ** is a point of attachment to ULM;
L₁ and L₂ are each independently a bond, CR^{1a}R^{1b}, O, S, SO, SO₂, NR^{1c}, SO₂NR^{1c}, SONR^{1c}, SO(═NR^{1c}), SO(═NR^{1c})NR^{1d}, CONR^{1c}, NR^{1c}CONR^{1d}, NR^{1c}C(O)O, NR^{1c}SO₂NR^{1d}, CO, CR^{1a}═CR^{1b}, C≡C, SiR^{1a}R^{1b}, P(O)R^{1a}, P(O)OR^{1a} (CR^{1a}R^{1b})₁₋₄, —(CR^{1a}R^{1b})₁₋₄O(CR^{1a}R^{1b})_{1- 4}, —(CR^{1a}R^{1b})₁₋₄S(CR^{1a}R^{1b})₁₋₄, —(CR^{1a}R^{1b})₁₋₄ NR^{1c}(CR^{1a}R^{1b})₁₋₄, NR^{1c}C(═NCN)NR^{1d}, NR^{1c}C(═NCN), NR^{1c}C(═CNO₂)NR^{1d};
ring A₁ and ring A₂ are each independently 3-11 membered cycloalkyl, optionally substituted with 1-8 R^{1a} or R^{1b} groups, 3-11 membered heterocyclyl optionally substituted with 1-8 R^{1a} or R^{1b} groups, aryl optionally substituted with 1-8 R^{1a} or R^{1b} groups, or heteroaryl optionally substituted with 1-8 R^{1a} or R^{1b} groups,
wherein R^{1a}, R^{1b}, R^{1c}, R^{1d} and R^{1e} are each independently, —H, -D, -halo, —C₁-C₈alkyl, —O—C₁-C₈alkyl, —C₁-C₆haloalkyl, —S—C₁-C₈alkyl, —NHC₁-C₈alkyl, —N(C₁-C₈alkyl)2, 3-11 membered cycloalkyl, aryl, heteroaryl, 3-11 membered heterocyclyl, —O-(3-11 membered cycloalkyl), —S-(3-11 membered cycloalkyl), NH-(3-11 membered cycloalkyl), N(3-11 membered cycloalkyl)₂, N-(3-11 membered cycloalkyl)(C₁-C₈alkyl), —OH, —NH₂, —SH, —SO₂C₁-C₈alkyl, —SO₂-aryl, —SO₂-heteroaryl, SO(NH)C₁-C₈alkyl, P(O)(OC₁-C₈alkyl)(C₁-C₈alkyl), —P(O)(OC₁-C₈alkyl)₂, —C≡C—C₁-C₈alkyl, —C≡CH, —CH═CH(C₁-C₈alkyl), —C(C₁-C₈alkyl)═CH(C₁-C₈alkyl), —C(C₁-C₈alkyl)═C(C₁-C₈alkyl)₂, —Si(OH)₃, —Si(C₁-C₈alkyl)₃, —Si(OH)(C₁-C₈alkyl)₂, —C(O)C₁-C₈alkyl, —C(O)OC₁-C₈alkyl, —CO₂H, —CN, —CF₃, —CHF₂, —CH₂F, —NO₂, —SF₅, —SO₂NHC₁-C₈alkyl, —SO₂N(C₁-C₈alkyl)₂, —SO(NH)NHC₁-C₈alkyl, —SO(NH)N(C₁-C₈alkyl)₂, —SONHC₁-C₈alkyl, —SON(C₁-C₈alkyl)₂, —CONHC₁-C₈alkyl, —CON(C₁-C₈alkyl)₂, —N(C₁-C₈alkyl)CONH(C₁-C₈alkyl), —N(C₁-C₈alkyl)-CON(C₁-C₈alkyl)₂, —NHCONH(C₁-C₈alkyl), —NHCON(C₁-C₈alkyl)₂, —NHCONH₂, —N(C₁-C₈alkyl)SO₂NH(C₁-C₈alkyl), —N(C₁-C₈alkyl)SO₂N(C₁-C₈alkyl)₂, —NHSO₂NH(C₁-C₈alkyl), —NHSO₂N(C₁-C₈alkyl)₂, or —NHSO₂NH₂; and where R^{1a} or R^{1b}, each independently may be optionally linked to other groups to form cycloalkyl and/or heterocyclyl moiety, optionally substituted with 1-4 R^{1e} groups.

16. The compound according to claim 15, wherein
L₁ is a bond, (C(R¹⁰)₂)ₚ, or CO;
L₂ is a bond, (C(R¹⁰)₂)ₚ, or CO;
p is 1, 2, 3 or 4;
each R¹⁰ is independently H or C₁-C₄ alkyl;
ring A₁ is a 3-7 membered cycloalkyl group, a 4-10-membered heterocycloalkyl group, an aryl group, or a heteroaryl group; and
ring A₂ is a 3-7 membered cycloalkyl group, a 4-10-membered heterocycloalkyl group, an aryl group, or a heteroaryl group.

17-19. (canceled)

20. The compound according to claim 15, wherein ring A₁ is a piperazine group, a morpholine group, a piperidine group, a pyrrolidine group, an azetidine group, or an azabicyclo-alkyl group.

21-22. (canceled)

23. The compound according to claim 15, wherein ring A₂ is a piperazine group, a morpholine group, a piperidine group, a pyrrolidine group, an azetidine group, a diazaspiroalkyl group or an azabicycloalkyl group.

24. (canceled)

25. The compound according to claim 15, wherein L is 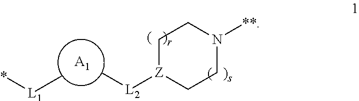
wherein
r is 0, 1 or 2;
s is 0, 1 or 2; and
Z is N or CR¹⁰.

26-28. (canceled)

29. The compound according to claim 15 that is a compound of formula II: 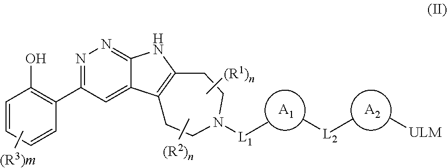
or a pharmaceutically acceptable salt thereof.

30. The compound according to claim 1, wherein ULM is 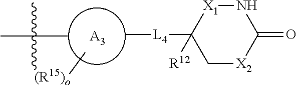
wherein: [symbol]
is a point of attachment to L;
Ring A₃ is a monocyclic, bicyclic or tricyclic aryl, heteroaryl, or heterocyclyl group,
L₄ is a bond, —O—, —S—, —NR^{a}—, —C(R^{a})₂—, or —C(O)NR^{a}—;
X₁ is CH₂, CO, CH═CH (when X₂═CO), or N═CH (when X₂═CO);
X₂ is CH₂, CO, CH═CH (when X₁═CO), or N═CH (when X₁═CO);
R¹² is H, optionally substituted C₁₋₄ alkyl, C₁₋₄ alkoxy, C₁₋₄haloalkyl, —CN, —OR^{a}, —OR^{b} or —SR^{b};
each R¹⁵ is independently H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b};
each R^{a} is independently H, —C(O)R^{b}, —C(O)OR^{c}, —C(O)NR^{c}R^{d}, —C(═NR^{b})NR^{b}R^{c}, —C(═NOR^{b})NR^{b}R^{c}, —C(═NCN)NR^{b}R^{c}, —P(OR^{c})₂, —P(O)R^{c}R^{b}, —P(O)OR^{c}OR^{b}, —S(O)R^{b}, —S(O)NR^{c}R^{d}, —S(O)₂R^{b}, —S(O)₂NR^{c}R^{d}, SiR^{b}₃, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl; or
R^{c} and R^{d}, together with the atom to which they are both attached, form a monocyclic or multicyclic heterocycloalkyl, or a monocyclic or multicyclic heterocycloalkenyl group; and
is 1, 2, 3, 4, or 5.

31-36. (canceled)

37. The compound according claim 30, wherein ULM is 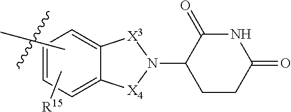
wherein: [symbol]
is a point of attachment to L;
X₃ is CH₂, CO, CH═CH (when X₄═CO), or N═CH (when X₄═CO); and
X₄ is CH₂, CO, CH═CH (when X₃═CO), or N═CH (when X₃═CO).

38-41. (canceled)

42. The compound according to claim 1, wherein ULM is 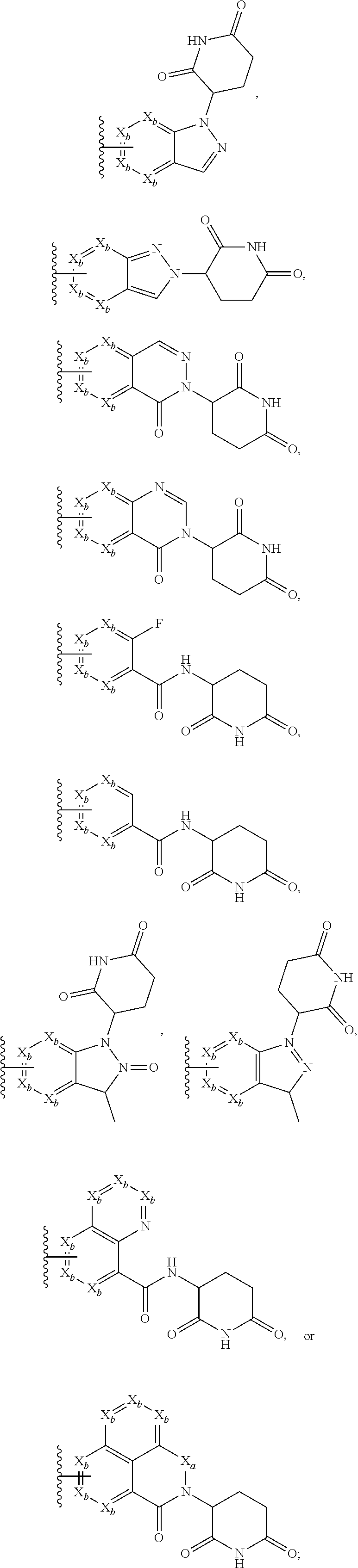
wherein [symbol] is a point of attachment to L;
Xₐ is a bond, —C(O)—, —C(S)—, —CH₂—, —CHCF₃—, SO₂—, —S(O), —P(O)R^{b}— or —P(O)OR^{b}—;
R^{b}, is H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl; and
each X_{b} is independently N or CR^{b}, provided that one X_{b} is a C atom having the attachment point to L.

43-70. (canceled)

71. The compound according to claim 15 that is a compound of formula III: 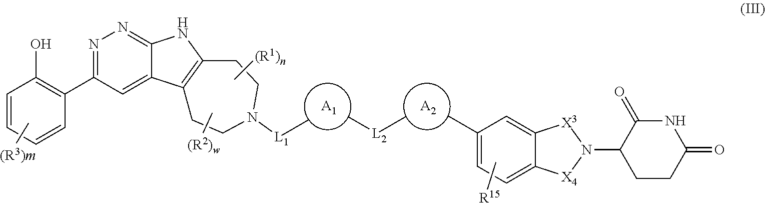
or a pharmaceutically acceptable salt thereof; wherein
L₁ is a bond, C(R¹⁰)₂, or CO;
L₂ is a bond, C(R¹⁰)₂, or CO;
each R¹⁰ is independently H or C₁-C₄ alkyl;
ring A₁ is a 3-11 membered heterocyclyl optionally substituted with 1-6 R^{1a} or R^{1b} groups;
ring A₂ is a 3-11 membered heterocyclyl optionally substituted with 1-6 R^{1a} or R^{1b} groups;
X³ is CH₂ or CO; and
X⁴ is CH₂ or CO; and
R¹⁵ is H, halogen, oxo, —OH, —CN, —NO₂, —C₁-C₆alkyl, —C₂-C₆alkenyl, —C₂-C₆alkynyl, C₀-C₁alk-aryl, C₀-C₁alk-heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{a}, —C(O)OR^{a}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{d})(OR^{c}) or —S(O)₂R^{b}.

72. The compound according to claim 71, wherein ring A₁ is a piperazine group, a morpholine group, a piperidine group, a pyrrolidine group, an azetidine group or an azabicyclo-alkyl group.

73. (canceled)

74. The compound according to claim 71, wherein ring A₂ is a piperazine group, a morpholine group, a piperidine group, a pyrrolidine group, an azetidine group, a diazaspiroalkyl group or an azabicycloalkyl group.

75. (canceled)

76. The compound according to claim 71 that is a compound of formula IV: 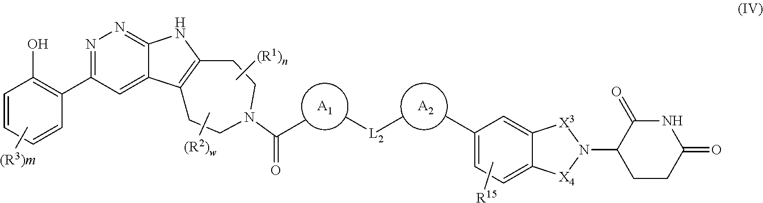
or a pharmaceutically acceptable salt thereof.

77. The compound according to claim 71 that is a compound of formula V: 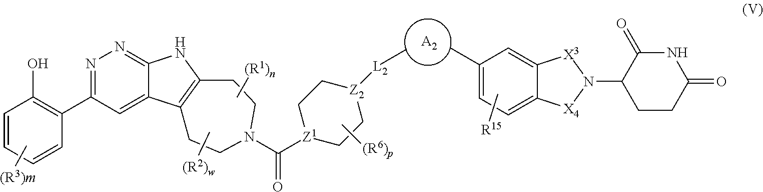
or a pharmaceutically acceptable salt thereof; wherein
Z₁ is N or CR⁶;
Z₂ is N or CR⁶;
each R⁶ is independently H, D, C₁₋₆ alkyl, C₃₋₆ cycloalkyl, or C₁₋₆ haloalkyl; and
p is 0, 1, 2, 3, 4, 5, 6, 7, or 8.

78-82. (canceled)

83. The compound according to claim 77 that is a compound of formula VI: 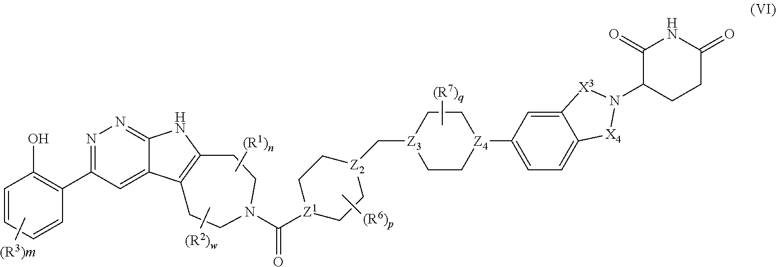
or a pharmaceutically acceptable salt thereof; wherein
Z₃ is N or CR₇;
Z₄ is N or CR⁷;
each R⁷ is independently H, D, C₁₋₆ alkyl, C₃₋₆ cycloalkyl, or C₁₋₆ haloalkyl; and
q is 0, 1, 2, 3, 4, 5, 6, 7 or 8.

84-88. (canceled)

89. The compound according to claim 83 that is a compound of formula VII: 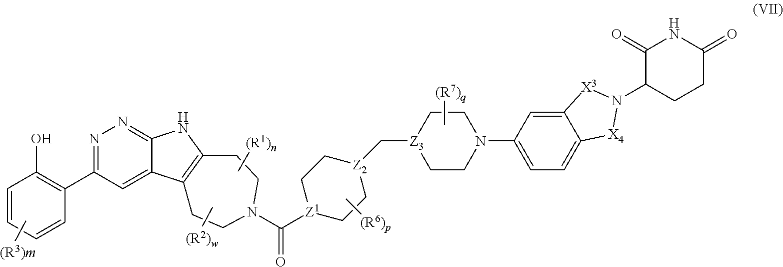
or a pharmaceutically acceptable salt thereof.

90-108. (canceled)

109. The compound according to claim 1 that is:
(S)-3-(6-(4-((4-(3-(2-Hydroxyphenyl)-5,6,7,8,9,10-hexahydropyridazino [4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione;
(S)-3-(6-(4-(((3R,5S)-4-(3-(2-hydroxyphenyl)-5,6,7,8,9,10-hexahydropyridazino [4′,3′:4,5]pyrrolo[2,3-d]azepine-7-carbonyl)-3,5-dimethylpiperazin-1-yl)methyl)piperidin-1-yl)-1-oxoisoindolin-2-yl)piperidine-2,6-dione;
(S)-3-(6-(4-((4-(3-(2-hydroxyphenyl)-5,6,7,8,9,10-hexahydropyridazino[4′,3′:4,5]pyrrolo[2,3-d]azepine-7-carbonyl)piperazin-1-yl)methyl)piperidin-1-yl)-1-oxoisoindolin-2-yl)piperidine-2,6-dione;
(S)-3-(6-(4-((4-(3-(3-Fluoro-2-hydroxyphenyl)-5,6,7,8,9,10-hexahydro-pyridazino [4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione;
(3S)-3-(6-(4-((4-(9-ethyl-3-(3-fluoro-2-hydroxyphenyl)-5,6,7,8,9,10-hexahydro-pyridazino[4′,3′:4,5]pyrrolo[2,3-d]azepine-7-carbonyl)-3,3-dimethylpiperazin-1-yl)methyl)piperidin-1-yl)-1-oxoisoindolin-2-yl)piperidine-2,6-dione;
(3S)-3-(6-(4-((4-(3-(3-fluoro-2-hydroxyphenyl)-6,7,8,9,10,11-hexahydro-5H-6,9-epiminocycloocta[4,5]pyrrolo[2,3-c]pyridazine-12-carbonyl)-3,3-dimethylpiperazin-1-yl)methyl)piperidin-1-yl)-1-oxoisoindolin-2-yl)piperidine-2,6-dione;
(3S)-3-(6-(4-((4-(3-(3-fluoro-2-hydroxyphenyl)-5,6,7,8,9,10-hexahydro-6,9-(epiminomethano)cyclohepta[4,5]pyrrolo[2,3-c]pyridazine-12-carbonyl)-3,3-dimethylpiperazin-1-yl)methyl)piperidin-1-yl)-1-oxoisoindolin-2-yl)piperidine-2,6-dione;
or a pharmaceutically acceptable salt thereof.

110. The compound of claim 1, in the form of a pharmaceutically acceptable salt.

111. A pharmaceutical composition comprising a compound according to claim 1, or a pharmaceutically acceptable salt thereof, and a pharmaceutically acceptable excipient.

112. A method of treating cancer in a subject in need thereof comprising administering to the subject a compound of claim 1 or a pharmaceutical composition comprising the compound.

113. The method of claim 112, wherein the cancer is SMARCA4 deleted cancer.

114. The method according claim 112, wherein the cancer is squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas.

115. The method according to claim 112, wherein the cancer is T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

116. The method of claim 115 wherein the lung cancer is SMARCA4 deficient non-small cell lung cancer.

117. A method of degrading a SMARCA protein comprising contacting the SMARCA protein with a compound of claim 1 or a pharmaceutical composition comprising the compound.
